(19) United States
(12) Patent Application Publication
(10) Pub. No.: US 2026/0042853 A1
(43) Pub. Date: Feb. 12, 2026

(54) BISPECIFIC IMMUNOTOXINS TARGETING HUMAN CD25+CCR4+ TUMORS AND REGULATORY T-CELLS

(71) Applicant: The Regents of the University of Colorado, a Body Corporate, Denver, CO (US)

(72) Inventors: Zhirui Wang, Denver, CO (US); Christene Huang, Denver, CO (US); David Mathes, Englewood, CO (US); Elizabeth Pomfret, Cherry Hills Village, CO (US)

(21) Appl. No.: 19/299,132

(22) Filed: Aug. 13, 2025

Related U.S. Application Data
(63) Continuation of application No. 17/431,853, now Pat. No. 12,421,316.
(60) Provisional application No. 62/807,676, filed on Feb. 19, 2019.

(51) Int. Cl. C07K 16/28; A61K 45/06; A61P 35/00; C07K 14/34; C07K 14/55

(52) U.S. Cl. CPC C07K 16/2866; A61K 45/06; A61P 35/00; C07K 14/34; C07K 14/55; C07K 2319/55

(57) ABSTRACT

IL2-CCR4 bispecific immunotoxin, CCR4-IL2 bispecific immunotoxin, and methods of use thereof for treatment of refractory and recurrent human CD25.sup.+ and/or CCR4.sup.+ cutaneous T cell lymphoma, and other human CD25.sup.+ or CCR4.sup.+ tumors. The bispecific immunotoxin can be also used for broad cancer treatment via depleting CD25.sup.+ or CCR4.sup.+ Tregs.

### CROSS-REFERENCE TO RELATED APPLICATION

This application is a continuation of U.S. application Ser. No. 17/431,853, filed Aug. 18, 2021, which in turn is a 371 application which claims benefit to PCT Application No. PCT/US2020/018922 filed Feb. 19, 2020 which claims the benefit of pending U.S. Provisional patent application No. 62/807,676, entitled “HUMAN IL2-CCR4 BISPECIFIC IMMUNOTOXIN TARGETING HUMAN CD25+CCR4+ TUMORS AND TREGS,” filed on Feb. 19, 2019, and is specifically incorporated by reference for all it discloses and teaches.

### REFERENCE TO A SEQUENCE LISTING

The application contains a Sequence Listing which has been submitted electronically in XML format and is hereby incorporated by reference in its entirety. Said XML copy, created on Sep. 22, 2025, is named P306999US02 and is 37,113 bytes in size.

### BACKGROUND

Cutaneous T-cell lymphoma (CTCL) is a type of extranodal non-Hodgkin's lymphoma characterized by skin lesions resulting from infiltration of malignant T lymphocytes. The two main forms of CTCL are Mycosis fungoides and Sezary syndrome. Treatment of early-stage CTCL (IA-IIA) primarily involves the use of skin directed therapies including topical corticosteroids, phototherapy, topical chemotherapy, topical bexarotene, and radiotherapy including localized radiation and total skin electron beam therapy. Refractory early-stage and advanced-stage CTCL (IIB-IV) requires systemic treatment using anti-cancer/chemotherapeutic agents such as bexarotene, vorinostat, denileukin diftitox, romidepsin, brentuximab and mogamulizumab. However, the objective response rate of these treatments remains approximately 30%.

### SUMMARY

The present invention is based on the development of bispecific immunotoxins that target the surface markers CC chemokine receptor 4 (“CCR4”) and/or IL-2 receptor α-chain (“CD25”), which are highly expressed by cutaneous T-cell lymphomas (CTCL). The efficacy of a CCR4 immunotoxin was compared to the efficacy of an IL2 fusion toxin, and the CCR4 immunotoxin was found to be more effective than the IL2 fusion toxin. Two bispecific immunotoxins were then constructed: 1) an IL2-CCR4 bispecific immunotoxin (SEQ ID NO: 4); and 2) a CCR4-IL-2 bispecific immunotoxin (SEQ ID NO: 2). The bispecific immunotoxins were expressed and purified using a diphtheria-toxin resistant yeast Pichia pastoris expression system. SDS-PAGE and Western Blot analysis demonstrated that the bispecific ^{˜}86 kDa.

The binding affinity of the biotinylated IL2-CCR4 bispecific immunotoxin and the CCR4-IL2 bispecific immunotoxin to human CD25.sup.+CCR4.sup.+Hut102/6TG was analyzed using flow cytometry. The bispecific immunotoxins showed higher in vitro binding affinity and efficacy than the monospecific immunotoxins. In vivo efficacy of the CCR4 immunotoxin, IL2 fusion toxin, and bispecific immunotoxins was assessed using CD25.sup.+CCR4.sup.+ CTCL Hut102/6TG-bearing immunodeficient NSG mouse model. The in vivo efficacy data demonstrated that CCR4 immunotoxin is more effective than IL2 fusion toxin and that the bispecific immunotoxins showed more efficacy and greater tumor response in vivo than either monospecific CCR4 immunotoxin or IL2 fusion toxin alone.

In some embodiments, the present invention includes a bispecific immunotoxin composed of a first part comprising a toxin, linked to a second part comprising the cytokine human interleukin-2 (IL-2), and a third part comprising an anti-human CC chemokine Receptor 4 (CCR4) antibody or fragment thereof, the second part being linked to the third part. In some embodiments, the second part is linked to the third part by a linker. In some embodiments, the first part is linked to the second part. In some embodiments, the first part is linked to the second part by a linker. In some embodiments, the first part is linked to the third part. In some embodiments, the first part is linked to the third part by a linker. In some embodiments, the toxin is diptheria toxin.

In some embodiments, the third part comprises at least one human CCR4-binding domain. In some embodiments, at least one human CCR4-binding domain comprises an antigen-binding portion of an anti-human CCR4 antibody. The antigen-binding portion of the anti-human CCR4 antibody may include VH and VL regions from an anti-human CCR4 antibody. In some embodiments the anti-human CCR4 antibody or fragment thereof is humanized. In some embodiments the anti-human CCR4 antibody is referred to as 1567 or mogamulizumab or KW-0761. In some embodiments, the 1567 may be mouse 1567 (SEQ ID NO: 6) or humanized 1567 (SEQ ID NO: 5), as presented in Chang, D K et al., Mol. Cancer Ther. 11, 2451-2461, 2012.

In some embodiments, the present invention includes a codon-optimized nucleic acid molecule optimized for expression in a methylotropic yeast encoding the bispecific immunotoxin.

In another aspect, the present invention includes a nucleic acid encoding the bispecific immunotoxin. In some embodiments, the present invention includes a vector comprising the nucleic acid molecule. In some embodiments, the present invention includes a host cell expressing the nucleic acid molecule. The host cell may be a methylotropic yeast. The host cell may be a cell of the species Pichia pastoris.

In some embodiments, the present invention includes a pharmaceutical composition comprising the bispecific immunotoxin and a physiologically acceptable carrier. The method may include administering to the subject a therapeutically effective amount of the bispecific immunotoxin. In some embodiments, the present invention comprises administering an immunotherapy to the subject.

In another aspect, the present invention includes a method of treating a subject who has a cancer. In some embodiments, the cancer is selected from the group of all CCR4.sup.+ and/or CD25+ tumors, and other tumors via regulatory T-cell (Tregs) depletion. In some embodiments the method of treating a subject who has a cancer may include administration of the bispecific immunotoxin in combination with another treatment including; surgery, radiation, chemotherapy, molecularly targeted therapy, immunotherapy, cell therapy or other standard of care.

In some embodiments, the cancer comprises cancer cells that express CD25. In some embodiments, the cancer comprises cancer cells that express CCR4. In some embodiments, the present invention comprises a method of depleting CD25-expressing Tregs in a subject. The method may include administering to the subject an effective amount of the bispecific immunotoxin. In some embodiments, the present invention comprises a method of depleting CCR4-expressing regulatory T cells in a subject. The method may include administering to the subject an effective amount of the bispecific immunotoxin.

In a further aspect, the present invention includes a method of producing an IL2-CCR4 bispecific immunotoxin. The method may include expressing a codon-optimized nucleic acid molecule encoding the bispecific immunotoxin of claim 1 in a methylotropic yeast, and substantially purifying the IL2-CCR4 bispecific immunotoxin, thereby producing the IL2-CCR4 bispecific immunotoxin. In some embodiments, the methylotropic yeast is Pichia pastoris.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1 is a schematic representation of four exemplary toxins in the disclosed technology: 1) a human IL2 fusion toxin; 2) a single-chain foldback diabody anti-human CCR4 immunotoxin; 3) an IL2-CCR4 bispecific immunotoxin; 4) a CCR4-IL2 bispecific immunotoxin.

FIG. 2 is an SDS-PAGE and Western blot analysis of the bispecific immunotoxins. A) SDS-PAGE analysis (4-12% NuPAGE, Invitrogen); The lower molecular weight weak band (˜45 kDa) in lane 2 is the broken down product of the IL2 fusion toxin; B) Western blot analysis using a mouse anti-His mAb (clone #: 4A12E4, Invitrogen); C) Western blot analysis using a mouse anti-diphtheria toxin mAb (clone #3B6, Meridian). Lane 1: Protein marker; Lane 2: monovalent human IL2 fusion toxin (59 kDa); Lane 3: single-chain foldback diabody anti-human CCR4 immunotoxin (96 kDa); Lane 4: IL2-CCR4 bispecific immunotoxin (86 kDa); Lane 5: CCR4-IL2 bispecific immunotoxin (86 kDa).

FIG. 3 is A) Flow cytometry binding affinity analysis of the biotinylated 1) IL2 fusion toxin alone; 2) CCR4 immunotoxin alone; 3) IL2-CCR4 bispecific immunotoxin; 4) CCR4-IL2 bispecific immunotoxin to human CD25.sup.+CCR4.sup.+Hut102/6TG cells. Fluorescein-Mouse anti human/rat CCR4 mAb and FITC-mouse anti-human CD25 mAb were used as positive controls. Biotin-labeled porcine CD3-ϵγ (Peraino, J S et al., Cell. Immunol. 276, 162-167, 2012) was included as a negative control for background due to protein biotinylation. The data are representative of three individual experiments. B) K.sub.D determination using flow cytometry and nonlinear least squares fit. MFI was plotted over a wide range of concentrations of biotinylated 1) IL2 fusion toxin alone; 2) CCR4 immunotoxin alone; 3) IL2-CCR4 bispecific immunotoxin; 4) CCR4-IL2 bispecific immunotoxin. The accompanying least-squares fits are shown based on the hyperbolic equation y=m.sub.1+m.sub.2*m.sub.0/(m.sub.3+m.sub.0) where y=MFI at the given biotinylated immunotoxin concentration, m.sub.0=biotinylated immunotoxin concentration, m.sub.1=MFI of zero biotinylated immunotoxin control, m.sub.2=MFI at saturation and m.sub.3=K.sub.D.

FIGS. 4A-4D is flow cytometry binding affinity analysis with data shown in panels. The left panel provides: Flow cytometry binding affinity analysis of the biotinylated IL2-CCR4 or CCR4-IL2 bispecific immunotoxins to A) human CD25 and CCR4 double negative Jurkat cell line; B) human CD25 single positive SR cell line; C-D) human CCR4 single positive CCL-119 cell line. Biotinylated IL2 fusion toxin alone, foldback-diabody anti-human CCR4 immunotoxin alone (CCR4 IT) and monovalent anti-human CCR4 immunotoxin alone (mono CCR4 IT) were included as controls. Fluorescein-mouse anti-human/rat CCR4 mAb and FITC-mouse anti-human CD25 mAb were used as positive controls. Biotin-labeled porcine CD3-ϵγ (Peraino et al., 2012) was included as a negative control for background due to protein biotinylation. The data are representative of three individual experiments. The right panel (in FIG. 4B-4D) provides: K.sub.D determination using flow cytometry and nonlinear regression, saturation binding equation by GraphPad Prism. MFI was plotted over a wide range of concentrations of the biotinylated 1) IL2 fusion toxin alone; 2) foldback diabody anti-human CCR4 immunotoxin alone (CCR4 IT); 3) IL2-CCR4 bispecific immunotoxin; 4) CCR4-IL2 bispecific immunotoxin; 5) monovalent anti-human CCR4 immunotoxin alone (Mono CCR4 IT, only in FIG. 4D). The nonlinear regression fit shown was based the equation Y=Bmax*X/(K.sub.D+X), where Y=MFI at the given biotinylated immunotoxin concentration after subtracting off the background; X=biotinylated immunotoxin concentration; Bmax=the maximum specific binding in the same units as Y.

FIG. 5 is in vitro efficacy analysis of the bispecific immunotoxins using CellTiter-Glo® Luminescent Cell Viability Assay (Promega, cat #G7571) to human CD25.sup.+CCR4.sup.+ T-cell lymphoma cell line Hut102/6TG. 1) C21 immunotoxin as negative control (C21 IT control); 2) IL2 fusion toxin alone (IL2 IT); 3) CCR4 immunotoxin alone (CCR4 IT); 4) IL2-CCR4 bispecific immunotoxin (IL2-CCR4 IT); 5) CCR4-IL2 bispecific immunotoxin (CCR4-IL2). Y-axis: inhibition rate of the cell viability by determining the number of viable cells based on the quantification of the ATP present. X-axis: plated immunotoxin concentration. Cycloheximide (1.25 mg/mL) was used as a positive control. The negative control contained cells without immunotoxin. Data are representative of multiple assays.

FIGS. 6A-6D reports an in vitro efficacy analysis of the bispecific immunotoxins using CellTiter-Glo® Luminescent Cell Viability Assay (Promega, cat #G7571) to A) human CD25 and CCR4 double negative Jurkat cell line; B) human CD25 single positive SR cell line; C and D) human CCR4 single positive CCL-119 cell line. C21 immunotoxin as negative control (FIG. 6A-C, C21 IT control); IL2 fusion toxin alone (FIG. 6A-C, IL2 IT); foldback diabody anti-human CCR4 immunotoxin alone (CCR4 IT) (FIG. 6A-C, CCR4 IT); IL2-CCR4 bispecific immunotoxin (FIG. 6A-D, IL2-CCR4 IT); CCR4-IL2 bispecific immunotoxin (FIG. 6A-D, CCR4-IL2 IT); monovalent anti-human CCR4 immunotoxin (mono CCR4 IT) (FIG. 6D, Mono CCR4 IT). Y-axis: inhibition rate of the cell viability by determining the number of viable cells based on the quantification of the ATP present. X-axis: plated immunotoxin concentration. Cycloheximide (1.25 mg/mL) was used as a positive control. The negative control contained cells without immunotoxin. Data are representative of multiple assays.

FIG. 7 is in vivo efficacy assessment of the bispecific immunotoxins. NSG mice were IV injected with 1.0×10.sup.7 human CD25.sup.+CCR4.sup.+ Hut102/6TG cells on day 0 and treated from day 4 on with the immunotoxin (IP injection) at 8.3×10.sup.-10 moles/kg daily for 10 consecutive days. 1) C21 immunotoxin control group (a non-related DT390-based immunotoxin as negative control) (n=13, C21 IT control) with a median survival time of 24 days; 2) IL2 fusion toxin alone group (n=12, IL2 IT) with a median survival time of 33.5 days; 3) CCR4 immunotoxin alone group (n=12, CCR4 IT) with median survival time of 40 days; 4) IL2-CCR4 bispecific immunotoxin group (n=12, IL2-CCR4 IT) with a median survival time of 57 days; 5) CCR4-IL2 bispecific immunotoxin group (n=14, CCR4-IL2 IT) with a median survival time of 69 days. The schedule of the immunotoxin and tumor cell injection is pictured in the schematic below the survival curve. The vertical arrows indicate the days on which the tumor cells or the immunotoxins were injected. The data are pooled from two separate experiments.

FIG. 8 is a liver necropsy examination of the representative tumor-bearing mice at day 21 from A) C21 immunotoxin group; B) IL2 fusion toxin alone group; C) CCR4 immunotoxin alone group; D) IL2-CCR4 bispecific immunotoxin group; E) CCR4-IL2 bispecific immunotoxin group.

FIG. 9 is a liver necropsy examination (repetition) of the representative tumor-bearing mice at day 21 from A) C21 immunotoxin group; B) IL2 fusion toxin alone group; C) CCR4 immunotoxin alone group; D) CCR4-IL2 bispecific immunotoxin group.

FIG. 10 is a pathology analysis: A) and F) Liver from a mouse injected with both Hut102/6TG tumor cells and C21 immunotoxin (negative control) shows extensive tumor infiltration with replacement of liver parenchyma by tumor cells. B) and G) Liver from a mouse injected with both Hut102/6TG tumor cells and IL2 fusion toxin shows fewer tumor cell areas in the examined section. C) and H) Liver from a mouse injected with both Hut102/6TG and CCR4 immunotoxin shows only a few sporadic tumor nodules. D-E) and I-J) Liver from a mouse injected with both Hut102/6TG and IL2-CCR4 or CCR4-IL2 bispecific immunotoxin shows normal or near normal hepatic parenchyma in the examined section. Two tumor cell areas were observed in the examined section of IL2-CCR4 immunotoxin treated animal liver (D) and none in CCR4-IL2 immunotoxin treated animal liver (E).

### DETAILED DESCRIPTION

The majority of clinically diagnosed cutaneous T-cell lymphomas (CTCL) highly express the cell surface markers CC chemokine receptor 4 (“CCR4”) and/or IL-2 receptor α-chain (“CD25”). CCR4 is overexpressed in CTCL skin lesions at all stages of disease and is recognized as a therapeutic target for CTCL (Sugaya, M., et al., J. Dermatol. 42, 1-3, 2015; Ferenczi, K. et al., J. Invest. Dermatol. 119, 1405-1410, 2002). Immunohistochemical CCR4 expression is 14-97% in the skin of CTCL patients and 90-100% in the clinical trials enrolling patients with relapsed diseases. CCR4 is detectable in almost all of the CTCL cases involving the blood using flow cytometry and percentages of CCR4 positive cells range from 31% to 97%. This is significantly higher than among healthy individuals (27%) (Ollila, T A, et al., Onco Targets Ther. 12, 1085-1094, 2019).

Although CD25 is expressed in fewer CTCL cases than CCR4, it is recognized as an important therapeutic target. Nichols, J. et al., Eur J Cancer 33 Suppl 1, S34-6, 1997 reported that CD25 is expressed in ˜50% of CTCL cases. CD25 expression is found more commonly in lesions from advanced CTCL patients (Talpur, R. et al., J Invest Dermatol. 126, 575-83, 2006).

Previously, a recombinant anti-human CCR4 immunotoxin for targeting CCR4.sup.+ tumors and Tregs was developed using a unique diphtheria toxin resistant yeast Pichia pastoris expression system (Wang, Z. et al., Mol. Oncol. 9, 1458-1470, 2015). The efficacy for targeting CCR4.sup.+ tumors was characterized using a CCR4.sup.+ T-cell acute lymphoblastic leukemia tumor-bearing immunodeficient NSG mouse model (Wang et al., 2015). The CCR4.sup.+ Treg depletion efficacy was demonstrated using naive cynomolgus monkeys (Wang, Z. et al., Mol. Oncol. 10, 553-565, 2016; Wang, Z. et al., Mol. Oncol. 12, 1374-1382, 2018). A human IL-2 fusion toxin (IL2 fusion toxin) was developed using a diphtheria toxin resistant yeast Pichia pastoris expression system (Liu, Y Y et al., Protein Expr. Purif 30, 262-274, 2003) and characterized its efficacy in vitro and in vivo (Peraino, J S et al., J. Immunol. Methods 405, 57-66, 2014; Wang, et al., J. Immunol. Methods 448, 51-58, 2017).

In the present invention, the efficacy of the human IL2 fusion toxin and the anti-human CCR4 immunotoxin described above were compared for targeting human CD25.sup.+CCR4.sup.+ CTCL. The CCR4 immunotoxin was more effective than the IL2 fusion toxin. Then, an IL2-CCR4 bispecific immunotoxin (SEQ ID NO: 4) and a CCR4-IL2 bispecific immunotoxin (SEQ ID NO: 2) were constructed. The bispecific immunotoxins were significantly more effective than either IL2 fusion toxin or CCR4 immunotoxin alone. The bispecific immunotoxins are targeted therapeutic drug candidates for the treatment of refractory and recurrent human CD25.sup.+ and/or CCR4.sup.+ CTCL.

The exemplary reagents constructed under this disclosure are generated by; 1) linking one, two or more, IL-2 polypeptides to a toxin, e.g., the truncated diphtheria toxin (DT390) for the human IL2 fusion toxin; 2) linking one or two anti-human CCR4 scFv polypeptides to a toxin, e.g., the truncated diphtheria toxin (DT390) for the CCR4 immunotoxin; and 3) linking IL-2 polypeptides, a toxin, and anti-human CCR4 scFv polypeptides, in varying order, for a bispecific immunotoxin.

In one embodiment (SEQ ID NO: 1), DT390 (SEQ ID NO: 7) was linked to anti-human CCR4 scFv, and the anti-human CCR4 scFv was linked to human IL2, as described below. DT390 (SEQ ID NO: 7) was linked to anti-human CCR4 scFv VL (SEQ ID NO: 9) via a linker sequence (SEQ ID NO: 8). Anti-human CCR4 scFv VL (SEQ ID NO: 9) was linked to anti-human CCR4 scFv VH (SEQ ID NO: 11) by a linker (SEQ ID NO: 10); and human IL2 (SEQ ID NO: 13) was linked to the anti-human CCR4 scFv VH (SEQ ID NO: 11) by a linker (SEQ ID NO: 12).

TABLE-US-00001 SEQ ID NO: 7:
GCT GGT GCT GAC
GACGTCGTCGACTCCTCCAAGTCCTTCGTCATGGAGAACTTCGCTTCCTACCACG
GGACCAAGCCAGGTTACGTCGACTCCATCCAGAAGGGTATCCAGAAGCCAAAGT
CCGGCACCCAAGGTAACTACGACGACGACTGGAAGGGGTTCTACTCCACCGACA
ACAAGTACGACGCTGCGGGATACTCTGTAGATAATGAAAACCCGCTCTCTGGAA
AAGCTGGAGGCGTGGTCAAGGTCACCTACCCAGGTCTGACTAAGGTCTTGGCTTT
GAAGGTCGACAACGCTGAGACCATCAAGAAGGAGTTGGGTTTGTCCTTGACTGA
GCCATTGATGGAGCAAGTCGGTACCGAAGAGTTCATCAAGAGATTCGGTGACGG
TGCTTCCAGAGTCGTCTTGTCCTTGCCATTCGCTGAGGGTTCTTCTAGCGTTGAAT
ATATTAATAACTGGGAACAGGCTAAGGCTTTGTCTGTTGAATTGGAGATTAACTT
CGAAACCAGAGGTAAGAGAGGTCAAGATGCGATGTATGAGTATATGGCTCAAGC
CTGTGCTGGTAACAGAGTCAGACGTTCTGTTGGTTCCTCTTTGTCCTGTATCAACC
TAGACTGGGACGTCATCAGAGACAAGACTAAGACCAAGATCGAGTCTTTGAAAG
AGCATGGCCCAATCAAGAACAAGATGTCCGAATCCCCCGCTAAGACCGTCTCCG
AGGAAAAGGCCAAGCAATACCTAGAAGAGTTCCACCAAACCGCCTTGGAGCATC
CTGAATTGTCAGAACTTAAAACCGTTACTGGGACCAATCCTGTATTCGCTGGGGC
TAACTATGCGGCGTGGGCAGTAAACGTTGCGCAAGTTATCGATAGCGAAACAGC
TGATAATTTGGAAAAGACAACTGCTGCTCTTTCGATACTTCCTGGTATCGGTAGC
GTAATGGGCATTGCAGACGGTGCCGTTCACCACAATACAGAAGAGATAGTGGCA
CAATCCATCGCTTTGTCCTCTTTGATGGTTGCTCAAGCTATCCCATTGGTCGGTGA
GTTGGTTGACATCGGTTTCGCTGCCTACAACTTCGTCGAGTCCATCATCAACTTGT
TCCAAGTCGTCCACAACTCCTACAACCGTCCGGCTTACTCCCCAGGTCACAAGAC
CCAACCATTCTTG CCA TGG
SEQ ID NO: 8:
GGT GGT GGT GGT TCT
SEQ ID NO: 9:
GAC ATT GAG TTG ACT CAA TCT CCA TCT TCC TTG GCT GTT TCT
GCT GGT GAG AAG GTT ACT ATG TCT TGT AAG TCT TCC CAA TCT ATT TTG
TAC TCT TCC AAC CAA AAG AAC TAC TTG GCT TGG TAC CAA CAA AAG CCA
GGT CAA TCT CCA AAG TTG TTG ATT TAC TGG GCT TCT ACT AGA GAG TCT
GGT GTT CCA GAC AGA TTC ACT GGT TCT GGT TCT GGT ACT GAC TTC ACT
TTG ACT ATT TCT TCC GTT CAA GCT GAG GAC TTG GCT GTT TAC TAC TGT
CAC CAA TAC TTG TCT TCC TAC ACT TTC GGT GGT GGT ACT AAG TTG GAG
ATT AAG
SEQ ID NO: 10:
GGT GGT GGT GGT TCT GGT GGT GGT GGA TCT GGT GGT GGT GGT TCT
SEQ ID NO: 11:
CAA GTT CAA TTG CAA CAA TCT GGT CCA GAG TTG GTT AGA
CCA GGT GCT TCT GTT AGA ATT TCT TGT AAG GCT TCT GGT TAC ACT TTC
GCT TCT TAC TAC ATT CAA TGG ATG AAG CAA AGA CCA GGT CAA GGT TTG
GAG TGG ATT GGT TGG ATT AAC CCA GGT AAC GTT AAC ACT AAG TAC AAC
GAG AAG TTC AAG GGT AAG GCT ACT TTG ACT GCT GAC AAG TCT TCC ACT
ACC GCT TAC ATG CAA TTG TCT TCC TTG ACT TCT GAG GAC TCT GCT GTT
TAC TTC TGT GCT AGA TCc ACT TAC TAC AGA CCA TTG GAC TAC TGG GGT
CAA GGT ACT ACC GTT ACT GTT TCT TCC
SEQ ID NO: 12:
GGT GGT GGT GGT TCT GGT GGT GGT GGA TCC GGT GGT GGT GGT TCT
SEQ ID NO: 13:
GCT CCA ACT TCT TCT TCT ACT AAG AAG ACT CAA TTG CAA
TTG GAG CAC TTG TTG TTG GAC TTG CAA ATG ATT TTG AAC GGT ATT AAC
AAC TAC AAG AAC CCA AAG TTG ACT AGA ATG TTG ACT TTC AAG TTC TAC
ATG CCA AAG AAG GCT ACT GAG TTG AAG CAC TTG CAA TGT TTG GAG GAG
GAA TTG AAG CCA TTG GAG GAA GTT TTG AAC TTG GCT CAA TCT AAG AAC
TTC CAC TTG AGA CCA AGA GAC TTG ATT TCT AAC ATT AAC GTT ATT GTT
TTG GAG TTG AAG GGT TCT GAG ACT ACT TTC ATG TGT GAG TAC GCT GAC
GAG ACT GCT ACT ATT GTT GAG TTC TTG AAC AGA TGG ATT ACT TTC TGT
CAA TCT ATT ATC TCT ACT TTG ACT CAC CAC CAC CAC CAC CAC

This sequence (SEQ ID NO: 1) encoded a human CCR4-IL2 bispecific immunotoxin (SEQ ID NO: 2):

TABLE-US-00002 (SEQ ID NO: 2):
AGADDVVDSSKSFVMENFASYHGTKPGYVDSIQKGIQKPKSGTQGNYDD
DWKGFYSTDNKYDAAGYSVDNENPLSGKAGGVVKVTYPGLTKVLALKVD
NAETIKKELGLSLTEPLMEQVGTEEFIKRFGDGASRVVLSLPFAEGSSS
VEYINNWEQAKALSVELEINFETRGKRGQDAMYEYMAQACAGNRVRRSV
GSSLSCINLDWDVIRDKTKTKIESLKEHGPIKNKMSESPAKTVSEEKAK
QYLEEFHQTALEHPELSELKTVTGTNPVFAGANYAAWAVNVAQVIDSET
ADNLEKTTAALSILPGIGSVMGIADGAVHHNTEEIVAQSIALSSLMVAQ
AIPLVGELVDIGFAAYNFVESIINLFQVVHNSYNRPAYSPGHKTQPFLP
WGGGGSDIELTQSPSSLAVSAGEKVTMSCKSSQSILYSSNQKNYLAWYQ
QKPGQSPKLLIYWASTRESGVPDRFTGSGSGTDFTLTISSVQAEDLAVY
YCHQYLSSYTFGGGTKLEIKGGGGSGGGGSGGGGSQVQLQQSGPELVRP
GASVRISCKASGYTFASYYIQWMKQRPGQGLEWIGWINPGNVNTKYNEK
FKGKATLTADKSSTTAYMQLSSLTSEDSAVYFCARSTYYRPLDYWGQGT
TVTVSSGGGGSGGGGSGGGGSAPTSSSTKKTQLQLEHLLLDLQMILNGI
NNYKNPKLTRMLTFKFYMPKKATELKHLQCLEEELKPLEEVLNLAQSKN
FHLRPRDLISNINVIVLELKGSETTFMCEYADETATIVEFLNRWITFCQ
SIISTLTHHHHRH

In another embodiment (SEQ ID NO: 3), DT390 (SEQ ID NO: 14) was linked to human IL2 (SEQ ID NO: 16); human IL2 (SEQ ID NO: 16) was linked to anti-human CCR4 scFv (SEQ ID NO: 18) by a linker (SEQ ID NO: 17), and the anti-human CCR4 scFv VL (SEQ ID NO: 18) was linked to anti-human CCR4 scFv VH (SEQ ID NO: 20) by a linker (SEQ ID NO: 19):

TABLE-US-00003 SEQ ID NO: 14: GCT GGT GCT GAC
GACGTCGTCGACTCCTCCAAGTCCTTCGTCATGGAGAACTTCGCTTCCT
ACCACGGGACCAAGCCAGGTTACGTCGACTCCATCCAGAAGGGTATCCA
GAAGCCAAAGTCCGGCACCCAAGGTAACTACGACGACGACTGGAAGGGG
TTCTACTCCACCGACAACAAGTACGACGCTGCGGGATACTCTGTAGATA
ATGAAAACCCGCTCTCTGGAAAAGCTGGAGGCGTGGTCAAGGTCACCTA
CCCAGGTCTGACTAAGGTCTTGGCTTTGAAGGTCGACAACGCTGAGACC
ATCAAGAAGGAGTTGGGTTTGTCCTTGACTGAGCCATTGATGGAGCAAG
TCGGTACCGAAGAGTTCATCAAGAGATTCGGTGACGGTGCTTCCAGAGT
CGTCTTGTCCTTGCCATTCGCTGAGGGTTCTTCTAGCGTTGAATATATT
AATAACTGGGAACAGGCTAAGGCTTTGTCTGTTGAATTGGAGATTAACT
TCGAAACCAGAGGTAAGAGAGGTCAAGATGCGATGTATGAGTATATGGC
TCAAGCCTGTGCTGGTAACAGAGTCAGACGTTCTGTTGGTTCCTCTTTG
TCCTGTATCAACCTAGACTGGGACGTCATCAGAGACAAGACTAAGACCA
AGATCGAGTCTTTGAAAGAGCATGGCCCAATCAAGAACAAGATGTCCGA
ATCCCCCGCTAAGACCGTCTCCGAGGAAAAGGCCAAGCAATACCTAGAA
GAGTTCCACCAAACCGCCTTGGAGCATCCTGAATTGTCAGAACTTAAAA
CCGTTACTGGGACCAATCCTGTATTCGCTGGGGCTAACTATGCGGCGTG
GGCAGTAAACGTTGCGCAAGTTATCGATAGCGAAACAGCTGATAATTTG
GAAAAGACAACTGCTGCTCTTTCGATACTTCCTGGTATCGGTAGCGTAA
TGGGCATTGCAGACGGTGCCGTTCACCACAATACAGAAGAGATAGTGGC
ACAATCCATCGCTTTGTCCTCTTTGATGGTTGCTCAAGCTATCCCATTG
GTCGGTGAGTTGGTTGACATCGGTTTCGCTGCCTACAACTTCGTCGAGT
CCATCATCAACTTGTTCCAAGTCGTCCACAACTCCTACAACCGTCCGGC
TTACTCCCCAGGTCACAAGACCCAACCATTCTTGCCA TGG
SEQ ID NO: 15: GGT GGT GGT GGT TCT
SEQ ID NO: 16: GCT CCA ACT TCT TCT TCT ACT AAG
AAG ACT CAA TTG CAA TTG GAG CAC TTG TTG TTG GAC
TTG CAA ATG ATT TTG AAC GGT ATT AAC AAC TAC AAG
AAC CCA AAG TTG ACT AGA ATG TTG ACT TTC AAG TTC
TAC ATG CCA AAG AAG GCT ACT GAG TTG AAG CAC TTG
CAA TGT TTG GAG GAG GAA TTG AAG CCA TTG GAG GAA
GTT TTG AAC TTG GCT CAA TCT AAG AAC TTC CAC TTG
AGA CCA AGA GAC TTG ATT TCT AAC ATT AAC GTT ATT
GTT TTG GAG TTG AAG GGT TCT GAG ACT ACT TTC ATG
TGT GAG TAC GCT GAC GAG ACT GCT ACT ATT GTT GAG
TTC TTG AAC AGA TGG ATT ACT TTC TGT CAA TCT ATT
ATC TCT ACT TTG ACT
SEQ ID NO: 17: GGT GGT GGT GGT
TCT GGT GGT GGT GGA TCC GGT GGT GGT GGT TCT
SEQ ID NO: 18: GAC ATT GAG TTG ACT CAA TCT CCA
TCT TCC TTG GCT GTT TCT GCT GGT GAG AAG GTT ACT
ATG TCT TGT AAG TCT TCC CAA TCT ATT TTG TAC TCT
TCC AAC CAA AAG AAC TAC TTG GCT TGG TAC CAA CAA
AAG CCA GGT CAA TCT CCA AAG TTG TTG ATT TAC TGG
GCT TCT ACT AGA GAG TCT GGT GTT CCA GAC AGA TTC
ACT GGT TCT GGT TCT GGT ACT GAC TTC ACT TTG ACT
ATT TCT TCC GTT CAA GCT GAG GAC TTG GCT GTT TAC
TAC TGT CAC CAA TAC TTG TCT TCC TAC ACT TTC GGT
GGT GGT ACT AAG TTG GAG ATT AAG
SEQ ID NO: 19: GGT GGT GGT GGT TCT GGT GGT GGT
GGA TCT GGT GGT GGT GGT TCT
SEQ ID NO: 20: CAA GTT CAA TTG CAA CAA TCT GGT
CCA GAG TTG GTT AGA CCA GGT GCT TCT GTT AGA ATT
TCT TGT AAG GCT TCT GGT TAC ACT TTC GCT TCT TAC
TAC ATT CAA TGG ATG AAG CAA AGA CCA GGT CAA GGT
TTG GAG TGG ATT GGT TGG ATT AAC CCA GGT AAC GTT
AAC ACT AAG TAC AAC GAG AAG TTC AAG GGT AAG GCT
ACT TTG ACT GCT GAC AAG TCT TCC ACT ACC GCT TAC
ATG CAA TTG TCT TCC TTG ACT TCT GAG GAC TCT GCT
GTT TAC TTC TGT GCT AGA TCc ACT TAC TAC AGA CCA
TTG GAC TAC TGG GGT CAA GGT ACT ACC GTT ACT GTT
TCT TCC CAC CAC CAC CAC CAC CAC

This sequence (SEQ ID NO: 3) encoded a human IL2-CCR4 bispecific immunotoxin (SEQ ID NO: 4):

TABLE-US-00004 SEQ ID NO: 4:
AGADDVVDSSKSFVMENFASYHGTKPGYVDSIQKGIQKPKSGTQGNYDD
DWKGFYSTDNKYDAAGYSVDNENPLSGKAGGVVKVTYPGLTKVLALKVD
NAETIKKELGLSLTEPLMEQVGTEEFIKRFGDGASRVVLSLPFAEGSSS
VEYINNWEQAKALSVELEINFETRGKRGQDAMYEYMAQACAGNRVRRSV
GSSLSCINLDWDVIRDKTKTKIESLKEHGPIKNKMSESPAKTVSEEKAK
QYLEEFHQTALEHPELSELKTVTGTNPVFAGANYAAWAVNVAQVIDSET
ADNLEKTTAALSILPGIGSVMGIADGAVHHNTEEIVAQSIALSSLMVAQ
AIPLVGELVDIGFAAYNFVESIINLFQVVHNSYNRPAYSPGHKTQPFLP
WGGGGSAPTSSSTKKTQLQLEHLLLDLQMILNGINNYKNPKLTRMLTFK
FYMPKKATELKHLQCLEEELKPLEEVLNLAQSKNFHLRPRDLISNINVI
VLELKGSETTFMCEYADETATIVEFLNRWITFCQSIISTLTGGGGSGGG
GSGGGGSDIELTQSPSSLAVSAGEKVTMSCKSSQSILYSSNQKNYLAWY
QQKPGQSPKLLIYWASTRESGVPDRFTGSGSGTDFTLTISSVQAEDLAV
YYCHQYLSSYTFGGGTKLEIKGGGGSGGGGSGGGGSQVQLQQSGPELVR
PGASVRISCKASGYTFASYYIQWMKQRPGQGLEWIGWINPGNVNTKYNE
KFKGKATLTADKSSTTAYMQLSSLTSEDSAVYFCARSTYYRPLDYWGQG
TTVTVSSHHHHHH

Without being limited to any one theory, these reagents are believed to function by binding to a cell surface via the IL-2/CD25 interaction and/or the anti-human CCR4 scFv/CCR4 receptor interaction; and the toxin, e.g., DT390 domain, is internalized by the cell. Following internalization, protein synthesis is inhibited, resulting in cell death.

The bispecific immunotoxins showed significantly higher efficacy for human CD25+ CCR4+ cells when compared to the monospecific immunotoxins alone. Without being limited to any one theory, linking human IL-2 fusion toxin and anti-human CCR4 scFv domains in tandem may increase the immunotoxins' affinity for IL-2 and human CCR4, facilitating a more efficient internalization, and causing an increase in potency. Endotoxin is a common concern when using an E. coli expression system for production. The present study utilized a diphtheria toxin-resistant yeast Pichia pastoris expression system (Liu, Y Y et al., 2003), which offers enhanced protein expression levels, purification and yield. Producing the bispecific immunotoxins in yeast rather than E. coli may augment the potential for clinical application of this reagent.

The truncated diphtheria toxin DT390 has been used to build recombinant immunotoxins (Woo et al., Protein Expr. Purif 25, 270-282, 2002; Kim et al., Protein Eng. Des. Sel. 20, 425-432, 2007; Wang et al., Bioconjug Chem. 22, 2014-2020, 2011). DT390 lacks the cell-surface binding domain and consists of the catalytic and translocation domains of the diphtheria toxin. In some embodiments, the toxin is diphtheria toxin (DT) or portions or variants thereof, e.g., Metl-Thr387, e.g., as described in Aullo et al, EMBO J. 11(2), 575-83, 1992; Abi-Habib et al, Blood 104(7), 2143-2148, 2004; Perentesis et al, Proc. Natl. Acad. Sci. USA 85, 8386-8390, 1988; Zettlemeissl et al, Gene. 41(1), 103-111, 1986; US 2009/0010966; US20090041797; U.S. Pat. Nos. 5,843,711; 7,585,942; 7,696,338; or US20080166375. In some embodiments, the toxin may be Pseudomonas exotoxin (PE), or portions or variants thereof, e.g., as described in U.S. Pat. Nos. 4,545,985; 4,892,827; 5,458,878; 7,314,632; Song et al, Protein Expression and Purification 44(1), 52-57, 2005; Theuer et al, J. Biol. Chem. 267(24), 16872-16877, 1992; Heimbrook et al, Proc Natl Acad Sci USA. 87(12), 4697-4701, 1990; Debinski et al, Mol Cell Biol. 11(3), 1751-1753, 1991; and Chaudhary et al, Proc. Nadl. Acad. Sci. USA 87, 308-312, 1990.

IL-2 binds to its cell surface receptor with notably strong affinity. The IL-2 receptor is a trimer composed of three subunits, α-β-γ. The a-subunit of this receptor, also known as CD25, is constitutively expressed on Tregs and has very high affinity for IL-2. There are species differences, which affect CD25 binding and subsequent target cell proliferation and differentiation (Zhang et al., Xenotransplantation 13, 423-32, 2006), thus it is important to match the IL-2 sequence used to the species of the subject to be treated (i.e., use the human IL-2, or a variant thereof (e.g., IL2 mutant) that binds the human IL-2 receptor, to treat human subjects).

Chemokine (C—C motif) receptor 4 (CCR4) is a G protein coupled receptor. It is a receptor for the chemokines, CCL17, and CCL22. CCR4 is constitutively expressed on Tregs and has a very low expression in most other cells of the immune system (Sugiyama et al., Proc Natl Acad Sci USA 110, 17945, 2013). Some human cancers (e.g., adult T-cell leukemia/lymphoma (Ishida et al., Cancer Sci. 97(11), 1139-1146, 2006); skin homing cutaneous T cell lymphoma (Ferenczi et al., J. Invest Dermatol 119(6), 1405-1410, 2002); acute T-cell lymphoblastic leukemia (Yoshie O. et al., Blood 99(5), 2002); Cutaneous T cell lymphoma/leukemia (CTCL), anaplastic large cell lymphoma (ALCL), peripheral T cell lymphoma (PTCL); and adult T-cell leukemia/lymphoma (ATLL) Yoshie, O et al., Int Immunol, epub, 2014) have also demonstrated high levels of CCR4 expression and can also be treated using methods described herein.

DNA and amino acid sequences are provided herein that include the linkers between DR390 and human IL2/anti-human CCR4 scFv, the linkers between human IL2 and anti-human CCR4 scFv, and the linkers between VL and VH.

The exemplary (G45).sub.3 linker used herein has been successfully used in following immunotoxins: anti-porcine CD3 immunotoxins (Wang et al., 2011); anti-human CD3 immunotoxin (Woo et al. 2002); anti-monkey CD3 immunotoxin (Kim et al, 2007).

Single chain variable fragments are fusion proteins of the variable regions of the heavy (VH) and light (VL) chains of immunoglobulins, connected into a single polypeptide chain with a short linker peptide (e.g., 1-50 or 10-25 amino acids). The linker allows the scFv to fold into a structure suitable for antigen binding. A monovalent construct includes a single ScFv. Two ScFvs can be linked, e.g., using a linker of 1-50, e.g., 10-25, amino acids, to form a divalent construct. Such divalent ScFv fusions include two VH and two VL domains with flexible linkers in between (e.g., VH-linker1-VL-linker2-VH-linker3-VL). In some embodiments, linkers 1 and 3 are the same, and are 10-25 amino acids long, and linker 2 is 15-20.

In some embodiments, the immunotoxins further include a peptide tag useful for purification. In some embodiments, the tag comprises histidines, e.g., two or more, e.g., three, four, five or six histidine residues at the C-terminus, and purification is achieved by binding to a nickel or cobalt column. In some embodiments, the tag comprises glutathione-S-transferase (GST) and recovery is by affinity to substrate glutathione bound to a column, e.g., glutathione sepharose.

The methods for producing immunotoxins described herein can be performed using protein production methods known in the art. For example, for scaled-up production, fermentation expression can be used.

In some embodiments, the methods use P. pastoris as a host organism, e.g., wild-type, X33, GS115 (his4), KM71, MC100-3, SMD1163, SMD1165, or SMD1168 strain. In other embodiments, other host organisms are contemplated.

Vectors suitable for use in the present methods are known in the art, and generally include a promoter, e.g., an AOX1, a constitutive P. pastoris promoter derived from the P. pastoris glyceraldehyde-3-phosphate dehydrogenase gene (GAP) promoter, typically followed immediately with a DNA sequence that encodes a secretion signal, e.g., the S. cerevisiae a factor prepro signal sequence, or the signal sequence derived from the P. pastoris acid phosphatase gene (PHO1).

The vectors can also include one or more yeast selectable markers that can be used to identify and/or select those cells that contain the vector can be used. Such markers can include drug resistance markers and pathways for synthesis of essential cellular components, e.g., nutrients. Drug resistance markers that can be used in yeast include chloramphenicol, kanamycin, methotrexate, G418 (geneticin), Zeocin, and the like. Markers in synthesis pathways can be used with available yeast strains having auxotrophic mutations in the corresponding gene; examples include the pathways for synthesizing leucine (LEU2), tryptophan (TRP1 and TRP2), proline (PRO1), uracil (URA3, URA5, URA6), histidine (HIS3), lysine (LYS2), adenine (ADEJ or ADE2), and the like. Other yeast selectable markers include the ARR3 gene from S. cerevisiae, which confers arsenite resistance to yeast cells that are grown in the presence of arsenate (Bobrowicz et al., Yeast 13, 819-828, 1997; Wysocki et al, J. Biol. Chem. 272, 30061-30066, 1997). A number of suitable integration sites include those enumerated in U.S. Pat. No. 7,479,389 and include homologs to loci known for Saccharomyces cerevisiae and other yeast or fungi. Methods for integrating vectors into yeast are well known (See for example, U.S. Pat. Nos. 7,479,389, 7,514,253, U.S. Published Application No. 2009012400, and WO2009/085135). Examples of insertion sites include, but are not limited to, Pichia ADE genes; Pichia TRP (including TRP J through TRP2) genes; Pichia MCA genes; Pichia CYM genes; Pichia PEP genes; Pichia PRB genes; and Pichia LEU genes. The Pichia ADE 1 and ARG4 genes have been described in Lin Cereghino et al, Gene 263, 159-169, 2001, and U.S. Pat. No. 4,818,700, the HIS3 and TRP1 genes have been described in Cosano et al, Yeast 14, 861-867, 1998, HIS4 has been described in GenBank Accession No. X5 180. See e.g., WO2011046855; Cregg, J M, Pichia Protocols 2(389), 2007; Romanos et al., Yeast 8, 423-488, 1992; Ilgen, et al., Gellissen, G. (ed.) (7), 143-16, 2004; Cereghino et al., FEMS Microbiology Reviews 24, 45-66 (2000); and Cregg, Research Corporation Technologies. Exemplary vectors include pPIC3K, pPIC9K, pA0815 and the pPICZ vector series.

Methods known in the art can be used for purification of the bispecific immunotoxins. For example, in one embodiment, a method may include a hexahistidine tag to facilitate purification, followed by ion exchange chromatography. In other embodiments, other methods, including ammonium sulfate precipitation, reversed phase chromatography, hydrophobic interaction chromatography (HIC), size exclusion chromatography, affinity chromatography, metal binding, immunoaffinity chromatography, HPLC, or purification tags (e.g., as described above) may be used to directly capture the purified proteins. See, e.g., Deutscher, M P, Methods in Enzymology, 1990; and The Recombinant Protein Handbook, Protein Amplification and Simple Purification, Amersham Pharmacia Biotech. After purification, the protein can optionally be concentrated, e.g., by lyophilization or ultrafiltration.

The bispecific immunotoxins described herein can be used in the treatment or study of certain disorders, e.g., blood cancers including; adult T-cell leukemia/lymphoma, skin homing cutaneous T cell lymphoma, acute T-cell lymphoblastic leukemia, cutaneous T cell lymphoma/leukemia, anaplastic large cell lymphoma, peripheral T cell lymphoma, and adult T-cell leukemia/lymphoma and cancers responsive to Treg depletion via CD25+ CCR4+ binding or characterized by CD25+ CCR4+ expression on the tumor cell surface. In some embodiments of this disclosure, the methods are used to treat subjects who have cutaneous T cell lymphoma.

In another embodiment, the bispecific immunotoxins described herein can also be used to target and deplete CD25+CCR4+ Treg cells. Treg cells suppress immune responses to tumors, therefore, methods that target and deplete this cell population in vivo could prove to be useful in improving cancer immunotherapy.

Generally, the methods include administering a therapeutically effective amount of bispecific immunotoxins as described herein, alone or in combination with another active or therapeutic agent, to a subject who is in need of, or who has been determined to be in need of, such treatment. In some embodiments, the methods also include administering one or more immunotherapies for cancer.

In some embodiments, the present invention provides a method of administering any of the disclosed compositions, including a therapeutically effective amount of the bispecific immunotoxins, described herein to a subject. When administered, the compositions are applied in a therapeutically effective, pharmaceutically acceptable amount as a pharmaceutically acceptable formulation.

As used herein, the term “pharmaceutically acceptable” is given its ordinary meaning. Pharmaceutically acceptable compositions are generally compatible with other materials of the formulation and are not generally deleterious to the subject. Any of the compositions of the present invention may be administered to the subject in a therapeutically effective dose. The dose to the subject may be such that a therapeutically effective amount of one or more other active compounds reaches the active site within the subject, for example via local or systemic administration. A “therapeutically effective” or an “effective” amount or dose, as used herein, means that amount necessary to delay the onset of, inhibit the progression of, halt altogether the onset or progression of, or otherwise achieve a medically desirable result, i.e., that amount which is capable of at least partially preventing, reversing, reducing, decreasing, ameliorating, or otherwise suppressing the particular condition being treated. A therapeutically effective amount can be determined on an individual basis and will be based, at least in part, on consideration of the species of subject's age, sex, size, and health; the composition used, the type of delivery system used; the time of administration relative to the severity of the disease; and whether a single, multiple, or controlled-release dose regiment is employed. A therapeutically effective amount can be determined by one of ordinary skill in the art employing such factors and using no more than routine experimentation.

The terms “treat,” “treated,” “treating,” and the like, when used herein, refer to administration of the systems and methods of the invention to a subject, which may, for example, increase the resistance of the subject to development or further development of cancers, to eliminate or at least control a cancer, and/or to reduce the severity of the cancer. The pharmaceutical preparations of the invention are administered to subjects in effective amounts. When administered to a subject, effective amounts will depend on the particular condition being treated and the desired outcome. A therapeutically effective dose may be determined by those of ordinary skill in the art.

In administering the systems and methods of the invention to a subject, dosing amounts, dosing schedules, routes of administration, and the like may be selected so as to affect known activities of these systems and methods. Dosage may be adjusted appropriately to achieve desired drug levels, local or systemic, depending upon the mode of administration.

The specific concentration may depend upon the particular composition used, as some are more effective than others. The dosage concentration of the composition actually administered is dependent, at least in part, upon the particular disorder being treated, the final concentration of composition that is desired at the site of action, the method of administration, the efficacy of the particular composition, the longevity of the particular composition, and the timing of administration relative to the severity of the disease. Preferably, the dosage form is such that it does not substantially deleteriously affect the subject.

The dosage may be given in some cases at the maximum amount while avoiding or minimizing any potentially detrimental side effects within the subject. The dosage actually administered can be dependent upon factors such as the final concentration desired at the active site, the method of administration to the subject, the efficacy of the composition, the longevity of the composition within the subject, the mode and/or timing of administration, the effect of concurrent treatments (e.g., as in a cocktail), etc. The dose delivered may also depend on conditions associated with the subject and can vary from subject to subject in some cases. For example, the age, sex, weight, size, environment, physical conditions, active site of the cancer or wound, or current state of health of the subject may also influence the dose required and/or the concentration of the composition at the active site. Variations in dosing may occur between different individuals or even within the same individual on different days. These factors are well known to those of ordinary skill in the art and can be addressed with no more than routine experimentation. It is preferred that a maximum dose be used, that is, the highest safe dose according to sound medical judgment.

Administration of a composition of the invention to a subject may be accomplished by any medically acceptable method which allows the composition to reach its target. The particular mode selected will depend of course, upon factors such as those previously described, for example, the particular composition, the severity of the state of the subject being treated, the dosage required for therapeutic efficacy, and route of administration etc.

The systems and methods of the invention can be administered by any method which allows the composition of the invention to reach the target cells, e.g., tumor cells. These methods include, e.g., injection, infusion, deposition, implantation, anal or vaginal supposition, oral ingestion, inhalation, topical administration, or any other method of administration where access to the target cells by the inhibitor is obtained.

In certain embodiments of the invention, the administration of the composition of the invention may be designed so as to result in sequential exposures to the composition over a certain time period, for example, hours, days, weeks, months, or years. This may be accomplished, for example, by repeated administration of a composition of the invention by one of the methods described above, and/or by a sustained or controlled release delivery system in which the composition is delivered over a prolonged period, usually without repeated administrations.

The following description provides specific embodiments. It is to be understood that other embodiments are contemplated and may be made without departing from the scope or spirit of the present invention. The following detailed description, therefore, is not to be taken in a limiting sense. While the present invention is not so limited, an appreciation of various aspects of the invention will be gained through a discussion of the examples provided below, which do not limit the scope of the invention described in the claims.

### EXAMPLES

The antibodies used in the Examples below are listed in Table 1.

TABLE-US-00005 TABLE 1
Antibodies used in this study Antibody Name Clone Source Cat#
Fluorescein-Mouse anti human/rat 205410 R&D FAB1567F
CCR4 Systems Mouse IgG2B
fluorescein isotype 133303 R&D IC0041F control Systems
FITC-mouse anti-human CD25
BC96 BioLegend 302604 FITC-mouse IgG1, κ isotype
control MOPC-21 BioLegend 400108
PE-streptavidin BioLegend 405204 Propidium Iodide Sigma
81845 7-Aminoactinomycin D (7-AAD) Sigma A9400

Human CD25.sup.+CCR4.sup.+ T-cell lymphoma cell line Hut 102/6TG (Williams D P, et al., J. Biol Chem 265, 20673-20677, 1990) were used in the examples.

Human T lymphocyte cell line, Jurkat, clone E6-1 (ATCC TIB-152); human CCR4.sup.+ acute lymphoblastic leukemia cell line CCRF-CEM (ATCC CCL-119); human CD25.sup.+ lymphoma cell line, SR (ATCC CRL-2262) were also used in the examples.

Protein expression in Pichia pastoris and subsequent purifications were performed as previously described (Wang et al., 2011; Example 1 and Peraino, J S et al., J. Immunol. Methods 398-399, 33-43, 2013). Western blot analysis, binding affinity and specificity analysis by flow cytometry and Kd determination were all performed as previously described (Example 1 and Peraino et al., 2013) using a human CD25+ T-cell lymphoma cell line HUT 102/6TG (William et al., 1990). DT390 and human IL-2 were used as controls for all in vitro functional analysis. These products were also expressed in the yeast Pichia pastoris system.

In vitro efficacy of the immunotoxins against the tumor cell line was assessed using CellTiter-Glo® Luminescent Cell Viability Assay (Promega, cat #G7571) as described previously (Zheng, Q. et al., Mol. Oncol. 11, 584-594, 2017). The cell viability assay measures the luminescence produced as a result of ATP production from metabolically active cells. The increasing concentrations of cytotoxic immunotoxins lead to cells death and a corresponding reduction in ATP related fluorescence. The luminescence signals were recorded using Wallac Victor2 1420 multilabel counter (Perkin Elmer).

Human CD25.sup.+CCR4.sup.+ CTCL Hut102/6TG tumor-bearing immunodeficient NSG mouse model was employed to assess the in vivo efficacy of the immunotoxins as described (Wang et al., 2015). Breeding pairs of immunodeficient NSG mice were purchased from Jackson laboratories (Bar Harbor, Me.) and bred in our rodent barrier facilities. The NSG mice were divided into the following groups: 1) C21 immunotoxin group as a negative control (a non-related diphtheria toxin-based immunotoxin) (n=13); 2) human IL2 fusion toxin group (n=12); 3) single-chain foldback diabody anti-human CCR4 immunotoxin group (n=12); 4) IL2-CCR4 bispecific immunotoxin group (n=12); 5) CCR4-IL2 bispecific immunotoxin group (n=14). All animals were IV injected at day 0 with 10 million human CD25.sup.+CCR4.sup.+ Hut102/6TG tumor cells via the tail vein. The immunotoxin was IP injected from day 4 on at 8.3×10.sup.-10 moles/kg, once daily for 10 consecutive days. The injected animals were observed daily for signs and symptoms of illness and scored at least twice weekly based on the parameters as previously reported by our lab (Peraino et al., 2013; Wang et al., 2015). The animals were humanely euthanized when the score exceeded the limit or the animal lost more than 15% of its pre-injection body weight.

Liver necropsy specimens were obtained surgically on day 21 after animal euthanasia. Tissues were fixed in 10% formalin and embedded in paraffin and subsequently sectioned. Tissues were stained with hematoxylin and eosin for routine light microscopy. Slides were digitalized by Aperio Scanscope (Leica), and images were analyzed at 2× and 30× with Aperio ImageScope software (Leica).

The survival curve comparison was performed using Log-rank (Mantel-Cox) test of GraphPad Prism 7 (GraphPad software Inc. San Diego, Calif.). IC.sub.50 was determined using nonlinear regression of Prism.

### Example 1

### Analysis of the Efficacy of CCR4 Immunotoxin Compared to IL2 Fusion Toxin to Human CD25.SUP.+.CCR4.SUP.+ CTCL Hut102/6TG

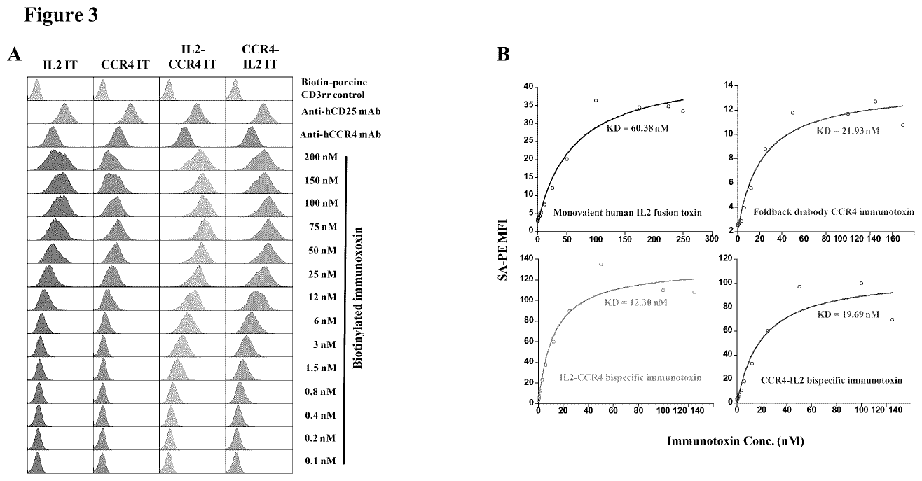

The binding affinity of the CCR4 immunotoxin to human CD25.sup.+ CCR4.sup.+ CTCL Hut102/6TG was analyzed using flow cytometry. The results showed that biotinylated CCR4 immunotoxin bound to human CD25.sup.+CCR4.sup.+ CTCL Hut102/6TG in a dose-dependent manner (FIG. 3A) with a Kd value of 21.93 nM (FIG. 3B), which was stronger than that of IL2 fusion toxin with Kd value of 60.38 nM (FIG. 3B). In vitro efficacy comparison of the CCR4 immunotoxin versus IL2 fusion toxin to human CD25.sup.+CCR4.sup.+ CTCL Hut102/6TG was performed using luminescent cell viability assay. As shown in FIG. 5 and Table 2, the IL2 fusion toxin (IC.sub.501=×10.sup.−10.5 M) was more effective than CCR4 immunotoxin (IC.sub.50=1×10.sup.−9.8 M) to human CD25.sup.+CCR4.sup.+ CTCL Hut102/6TG. In vivo efficacy comparison analysis demonstrated that CCR4 immunotoxin prolonged human CD25.sup.+CCR4.sup.+ Hut102/6TG-bearing immunodeficient NSG mouse survival significantly more than IL2 fusion toxin with median survival days of 40 versus 33.5 (FIG. 7, Tables 2 and 3).

TABLE-US-00006 TABLE 2
In vitro and in vivo efficacy summary of the tested immunotoxins
in this studyC21 IL2 CCR4 IL2- CCR4- IT IT IT CCR4 IT IL2
IT IC.sub.50 (M) .sup.
10.sup.-10.5 10.sup.-9.8 10.sup.-11.2 10.sup.-11.5 Median
24 33.5 40.sup. 57.sup. 69.sup. Survival Days

TABLE-US-00007 TABLE 3
Survival curve log-rank (Mantel-Cox) test C21 IL2 CCR4 IL2-
CCR4- IT IT IT CCR4 IT IL2 IT C21 IT — *** *** *** ***
IL2 IT *** — *** *** *** CCR4
IT *** *** — *** *** IL2-CCR4 IT *** *** *** — **
CCR4-IL2 IT *** *** *** ** — **p ≤ 0.01; ***p ≤ 0.001

### Example 2

### Expression and Purification of the IL2-CCR4 and CCR4-IL2 Bispecific Immunotoxins

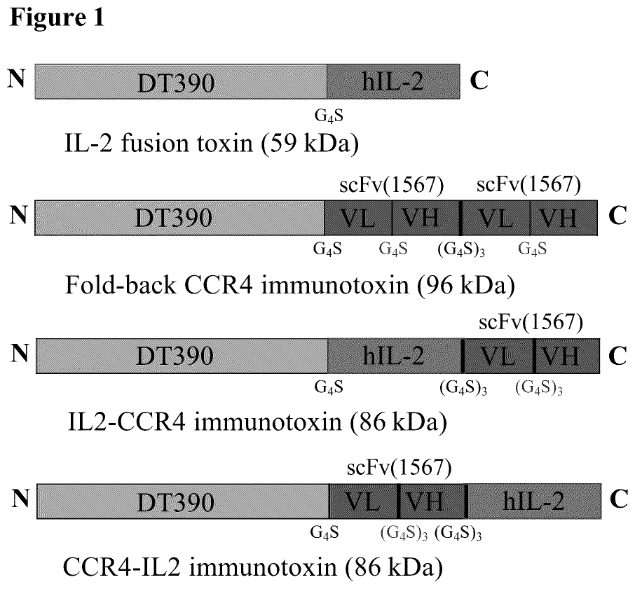

Two bispecific immunotoxins were constructed: 1) IL2-CCR4 bispecific immunotoxin; and 2) CCR4-IL2 bispecific immunotoxin. The bivalent anti-human CCR4 immunotoxin (Wang et al., 2015) was used as a template to construct the IL2-CCR4 or CCR4-IL2 bispecific immunotoxin by replacing the first or second anti-human CCR4 scFv using human IL2, as shown in FIG. 1, and described below.

IL2-CCR4 bispecific immunotoxin construction: bivalent anti-human CCR4 immunotoxin [DT390-BiscFv(1567)-6×His] in pwPICZalpha (Wang et al., 2015) was digested using NcoI and BamHI and separated with DNA agarose gel. The large band (˜4.35 kb) was cut out and extracted as vector. Bivalent human IL2 fusion toxin (DT390-Bi-hIL2-6×His) developed previously (Peraino et al, 2014) was digested using NcoI and BamHI. The digestion mixture was separated with DNA agarose gel. The human IL2 (˜459 bp) carrying NcoI and BamHI was cut out and extracted as insert. The prepared human IL2 insert carrying NcoI and BamHI was cloned into the prepared pwPICZalpha-DT390-scFv(1567) vector (NcoI-BamHI digested) yielding the IL2-CCR4 bispecific immunotoxin DNA construct (FIG. 1). Alternate vectors are available, including from Invitrogen.

CCR4-IL2 bispecific immunotoxin construction: bivalent anti-human CCR4 immunotoxin [DT390-BiscFv(1567)-6×His] in pwPICZalpha (Wang et al., 2015) was digested using BamHI and EcoRI and separated with DNA agarose gel. The large band (˜4.35 kb) were cut out and extracted as vector. Bivalent human IL-2 fusion toxin in pwPICZalpha (DT390-Bi-hIL2-6×His) (Peraino et al., 2014) was digested using BamHI and EcoRI. The digestion mixture was separated with DNA agarose gel. The human IL2 (−459 bp) carrying BamHI and EcoRI was cut out and extracted as insert. The prepared human IL2 insert carrying BamHI and EcoRI was cloned into prepared pwPICZalpha-DT390-scFv(1567) vector (BamHI-EcoRI digested) yielding the CCR4-IL2 bispecific immunotoxin DNA construct (FIG. 1). Alternate vectors are available, including from Invitrogen.

The linearized immunotoxin DNA constructs (IL2-CCR4 bispecific immunotoxin and the CCR4-IL2 bispecific immunotoxin) were transformed into diphtheria toxin resistant yeast Pichia pastoris strain (Liu et al., 2003) for expression and purification, as previously described (Wang et al., 2015). The strain was obtained by Angimmune.

IL2 fusion toxin (Peraino et al., 2014), CCR4 immunotoxin (Wang et al., 2015) and C21 immunotoxin (truncated diphtheria toxin DT390-based unrelated immunotoxin) were also expressed and purified in our lab using the same diphtheria toxin resistant yeast Pichia pastoris expression system. Western blot analysis, flow cytometry binding affinity analysis and Kd determination were all performed as previously described (Peraino et al., 2014; Wang et al., 2015). Methods for the yeast expression system are known (e.g., U.S. Pat. No. 7,892,786).

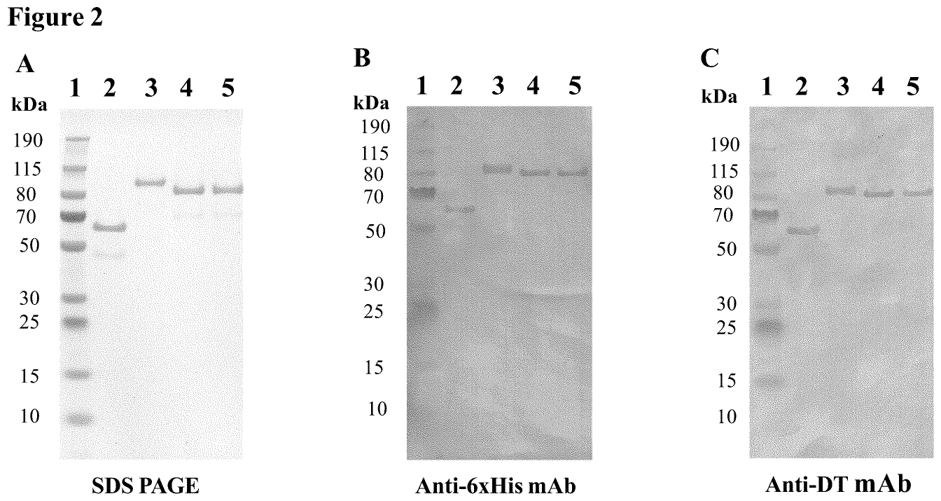

The final purification yield is ˜7 mg per liter of the original harvested supernatant. SDS-PAGE and Western Blot analysis demonstrated that the two versions of the bispecific immunotoxin were successfully expressed and purified with expected molecular weight of ˜86 kDa (FIG. 2A-C).

### Example 3

### Analysis of In Vitro Binding Affinity and Efficacy of Bispecific Immunotoxins Compared to Monospecific Immunotoxins

The binding affinity of the biotinylated IL2-CCR4 or CCR4-IL2 bispecific immunotoxin to human CD25.sup.+CCR4.sup.+ Hut102/6TG was analyzed using flow cytometry and found to have a Kd value of 12.3 nM for IL2-CCR4 immunotoxin and 19.69 nM for CCR4-IL2 immunotoxin (FIG. 3A-B). The bispecific immunotoxins bound more strongly than IL2 fusion toxin and CCR4 immunotoxin individually (FIG. 3A-B). The in vitro efficacy was assessed using luminescent based cell viability assay. The bispecific immunotoxins (IC.sub.50=10.sup.-11.2 M for IL2-CCR4 immunotoxin and IC.sub.50=10.sup.-11.5 M for CCR4-IL2 immunotoxin) were also more potent than either CCR4 immunotoxin (IC.sub.50=10.sup.-9.8 M) or IL2 fusion toxin alone (IC.sub.50=10.sup.-10.5 M) (FIG. 5 and Table 2).

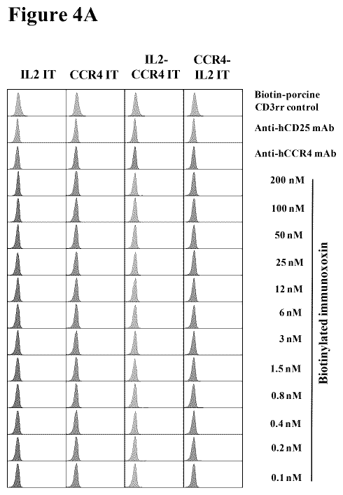

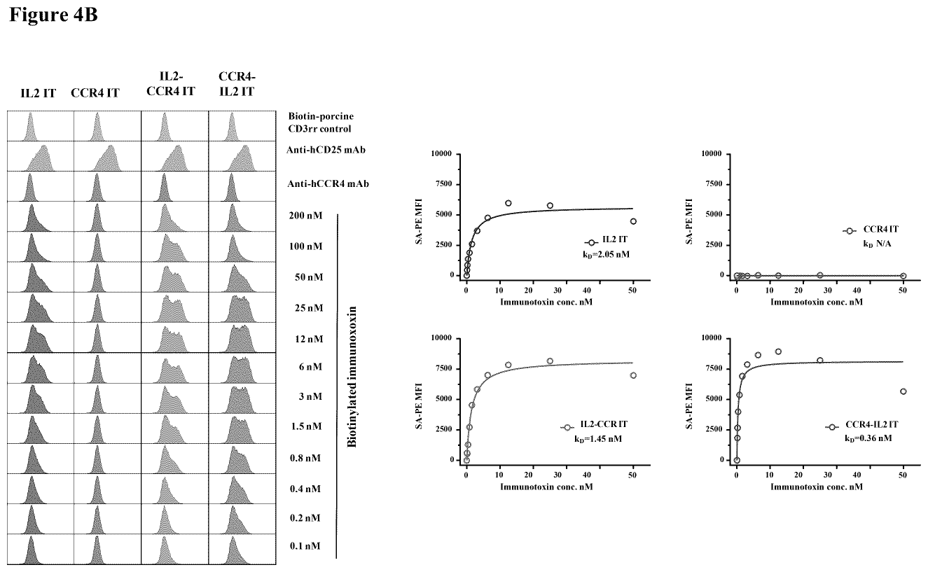

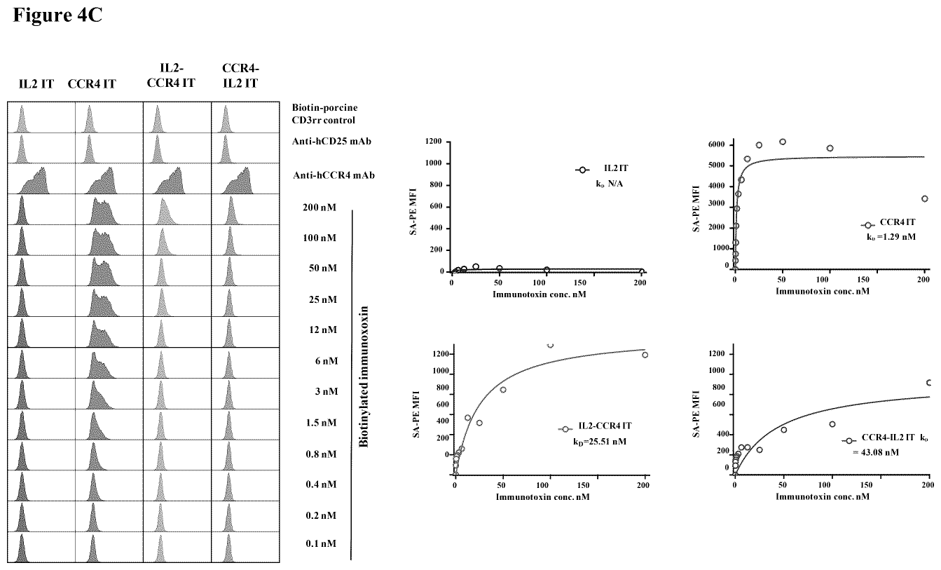

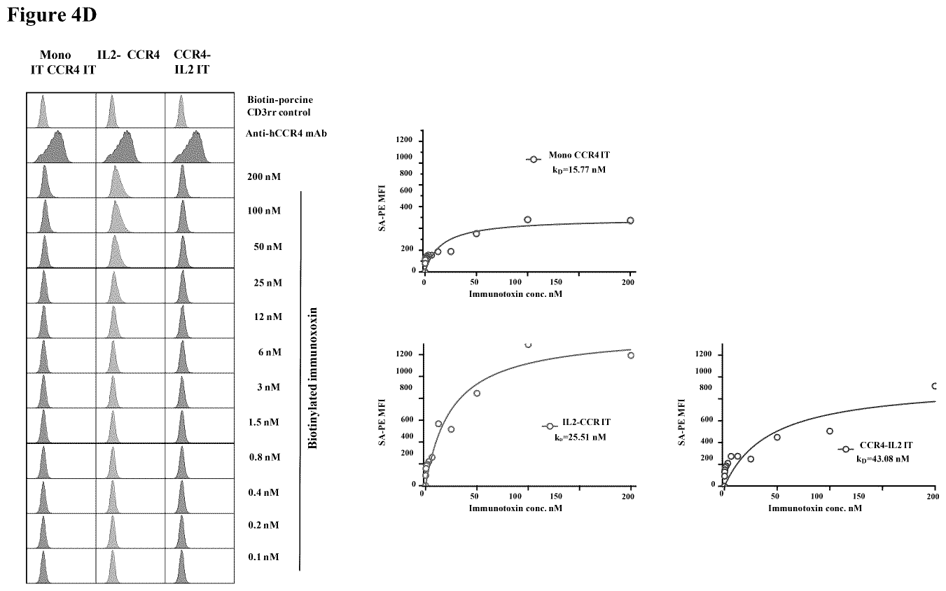

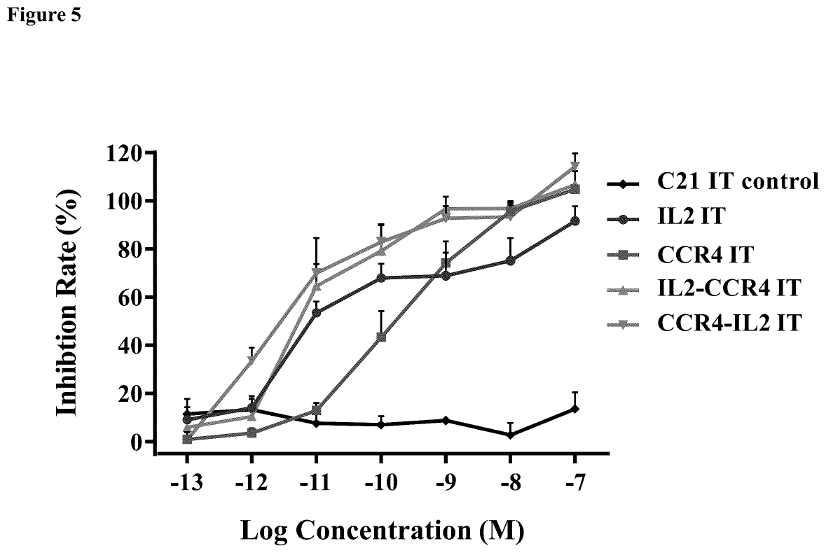

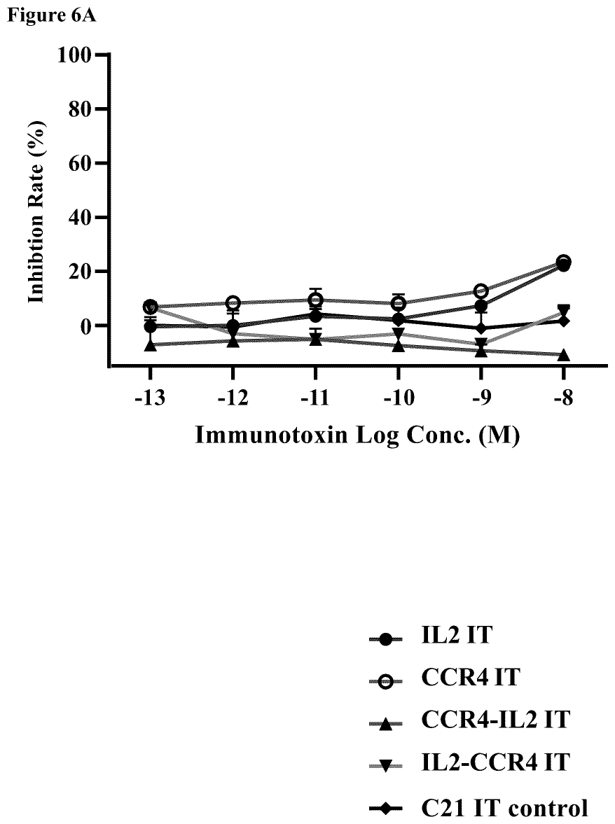

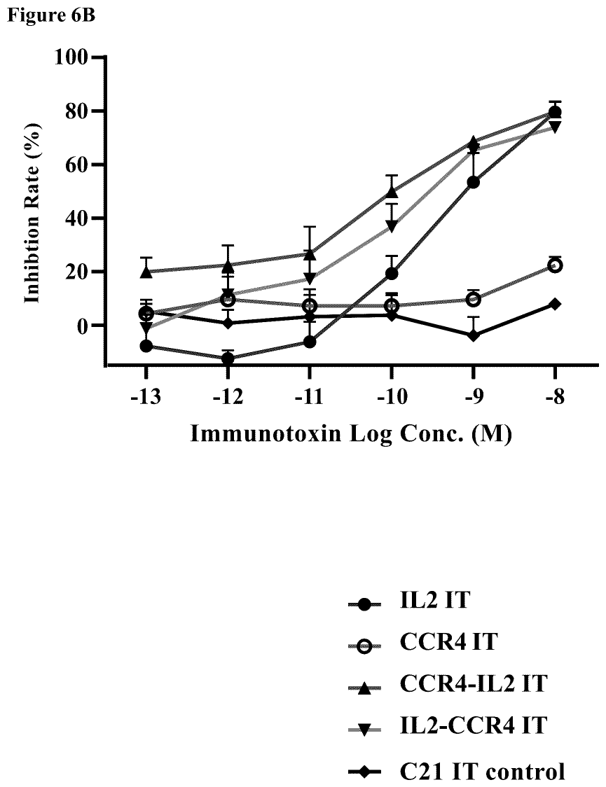

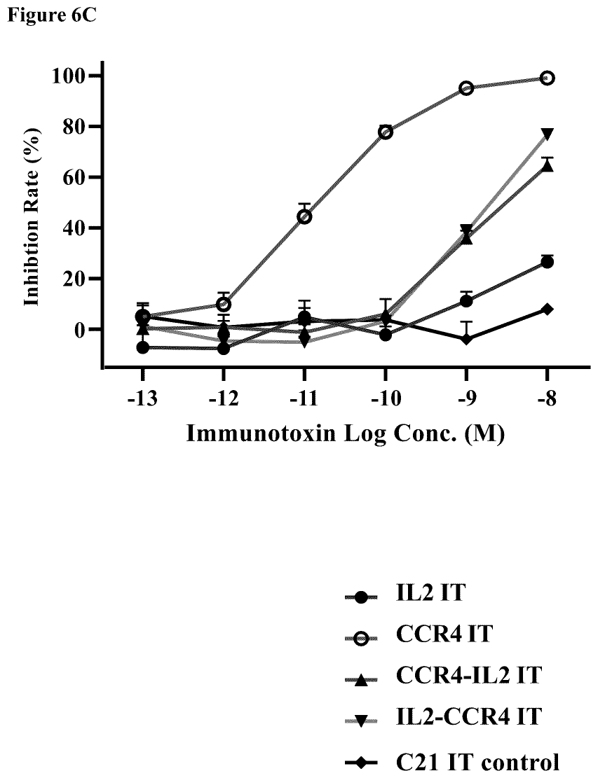

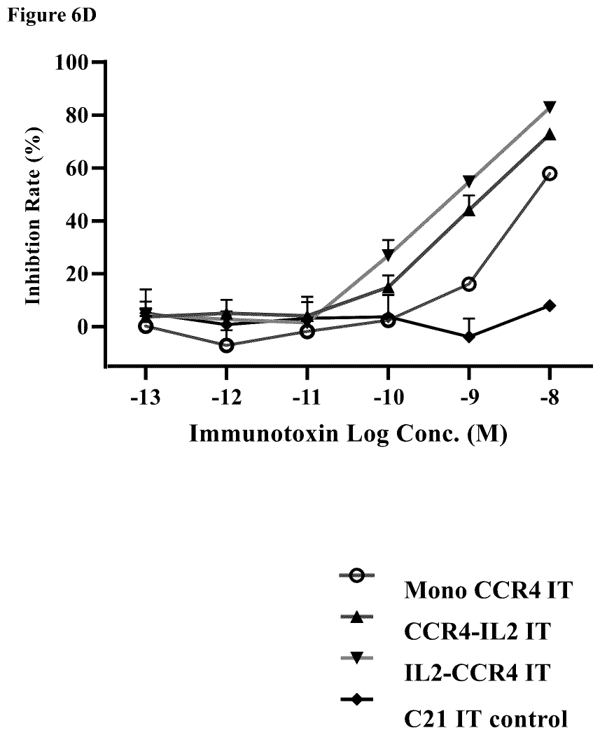

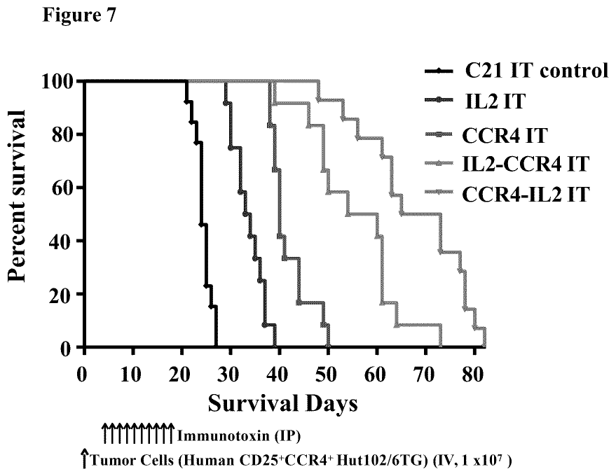

To further characterize the bispecific immunotoxins, in vitro flow cytometry binding affinity and luminescent based cell viability analysis of the bispecific immunotoxins was performed to 1) human CD25 and CCR4 double negative Jurkat cell line; 2) human CD25 single positive SR cell line; and 3) human CCR4 single positive CCL-119 cell line. As shown in FIGS. 4A and 6A, no in vitro binding or efficacy were observed for the bispecific immunotoxins to CD25 and CCR4 double negative Jurkat cells. As shown in FIGS. 4B and 6B, higher in vitro binding and efficacy were observed for the bispecific immunotoxins to CD25 single positive SR cells than that of the IL2 fusion toxin alone. As shown in FIGS. 4C and 4D, weak in vitro binding and efficacy were observed for the bispecific immunotoxins to CCR4 single positive CCL-119 cells. The binding affinity and efficacy were significantly weaker than that of the foldback diabody anti-human CCR4 immunotoxin (CCR4 IT) (FIGS. 4C and 6C, Wang et al., 2015) and comparable to that of the monovalent anti-human CCR4 immunotoxin (Mono CCR4 IT) (FIGS. 4D and 6D, Wang et al., 2015). Without being limited to any one theory, this may be because the bispecific immunotoxins only contain one anti-human CCR4 scFv. Taken together, the bispecific immunotoxins were more effective than IL2 fusion toxin or CCR4 immunotoxin alone to CCR4 and CD25 double positive or CD25 single positive cells, but less effective than foldback diabody anti-human CCR4 immunotoxin alone to CCR4 single positive cells. Foldback diabody anti-human CCR4 immunotoxin (Wang et al., 2015) is still more effective than the bispecific immunotoxins for targeting CCR4 single positive cells.

### Example 4

### Analysis of In Vivo Efficacy of the IL2 Bispecific Immunotoxins Using CD25.SUP.+.CCR4.SUP.+ CTCL Hut102/6TG-Bearing Immunodeficient NSG Mouse Model

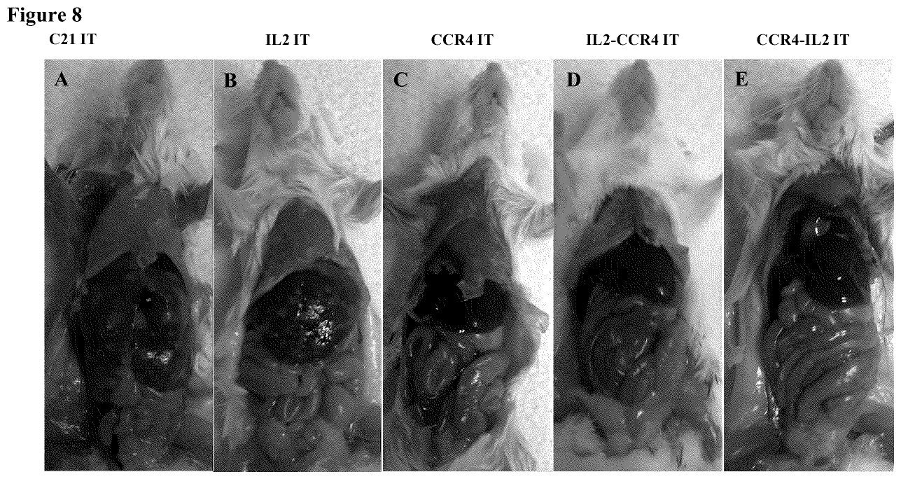

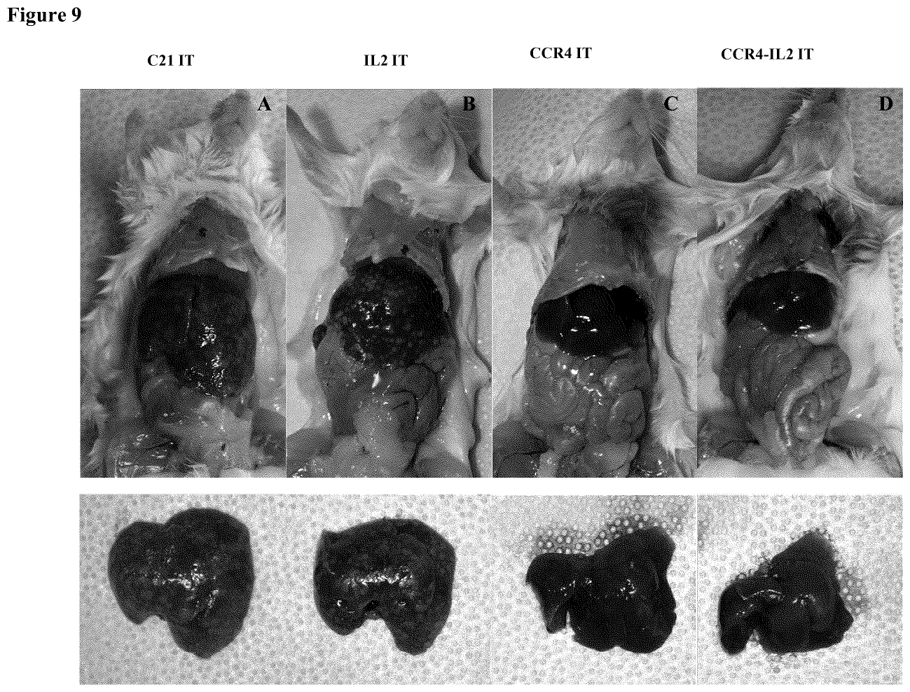

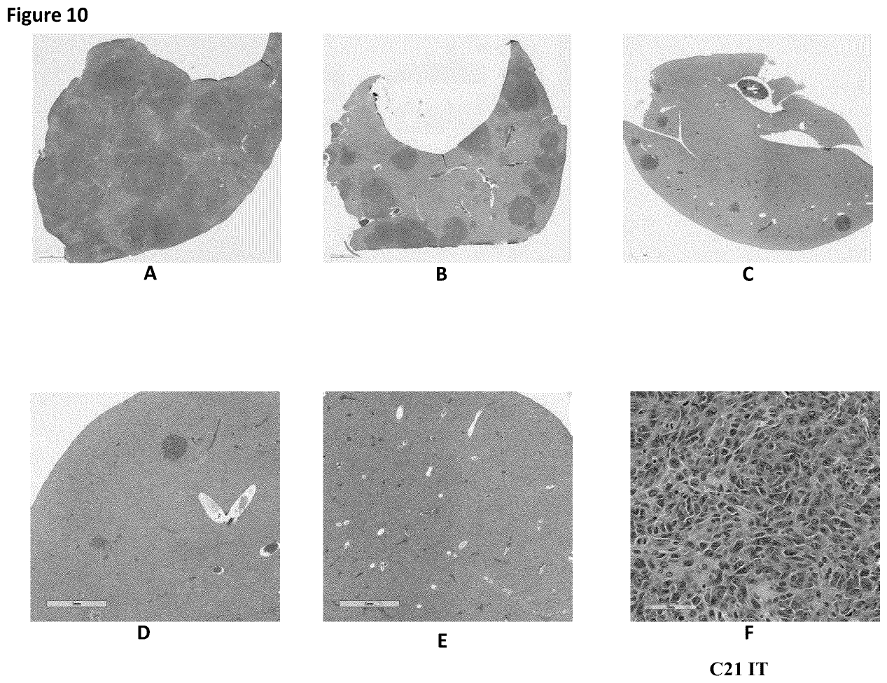

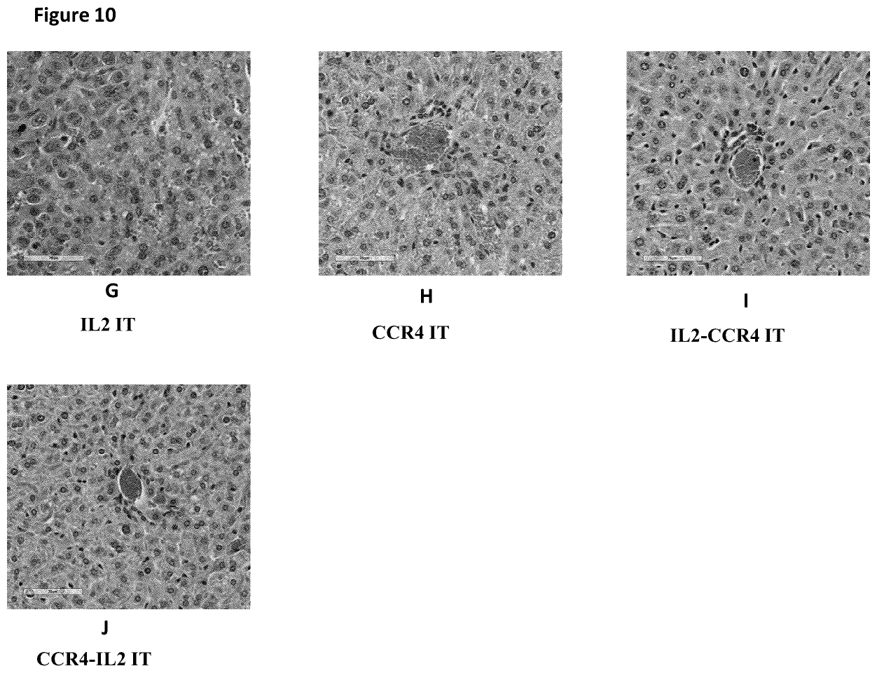

In vivo efficacy of the IL2-CCR4 or CCR4-IL2 bispecific immunotoxin was assessed using CD25.sup.+CCR4.sup.+ CTCL Hut102/6TG-bearing immunodeficient NSG mouse model. The tumor cells were intravenously (IV) injected on day 0. The immunotoxins were intraperitoneal (IP) injected starting on day 4 for 10 consecutive days. As shown in FIG. 7, the bispecific immunotoxins significantly prolonged median survival from 24 days in the C21 immunotoxin control group to 57 and 69 days in the IL2-CCR4 and CCR4-IL2 bispecific immunotoxin groups, respectively. This median survival was significantly longer than the median survival of 33.5 and 40 days for IL2 fusion toxin and CCR4 immunotoxin, respectively (FIG. 7, Table 2 and 3). As shown in FIG. 8 and FIG. 9, gross examination during necropsy on day 21 revealed enlarged livers with extensive CTCL tumor nodules on the liver surface of C21 immunotoxin control animals. The IL2 fusion toxin group had less hepatomegaly with fewer tumor nodules when compared with the C21 immunotoxin control group. The CCR4 immunotoxin group and the two bispecific immunotoxin groups demonstrated normal liver size with few sporadic tumor nodules (FIG. 8 and FIG. 9). Histological evaluation of the liver was performed at day 21 for five representative tumor-bearing NSG mice. For the C21 immunotoxin group, hematoxylin and eosin staining revealed replacement of liver parenchyma with extensive tumor nodules. (FIGS. 10A and 10F). In the IL-2 fusion toxin group, fewer tumor cell areas were seen in the examined section of the liver compared to that of the C21 immunotoxin group (FIGS. 10B and 10G). This pathology analysis confirms that the IL2 fusion toxin has efficacy in depletion of human CD25.sup.+CCR4.sup.+ tumor cells. Pathology of the CCR4 immunotoxin alone group showed sporadic tumor cell areas (FIGS. 10C and 10H), demonstrating that CCR4 immunotoxin treatment is more effective against the tumor cells than IL2 fusion toxin treatment. In the IL2-CCR4 immunotoxin group (FIGS. 10D and 10I, two tumor cell areas were identified in the examined section of the liver (see FIG. 10D). In the CCR4-IL2 immunotoxin group (FIGS. 10E and 10J), no tumor cell areas were identified in the examined section of the liver (see FIG. 10E), demonstrating that both the IL2-CCR4 and CCR4-IL2 bispecific immunotoxin more effectively depleted the human CD25.sup.+CCR4.sup.+ tumor cells than IL2 fusion toxin or CCR4 immunotoxin. Taken together, these in vivo efficacy data consistently demonstrated that CCR4 immunotoxin is more effective than IL2 fusion toxin and that the bispecific immunotoxin showed more efficacy and greater tumor response in vivo than either monospecific CCR4 immunotoxin or IL2 fusion toxin alone.

As shown in FIG. 7, Table 2 and 3, CCR4-IL2 bispecific immunotoxin prolonged the tumor-bearing animal survival significantly longer than IL2-CCR4 bispecific immunotoxin (median survival days of 69 vs 57). The only difference between these two bispecific immunotoxins is the order of IL2 and anti-human CCR4 scFv in the C-terminus of DT390 (either IL2-CCR4 or CCR4-IL2) (see FIG. 1). Histologic analysis demonstrated that two tumor cell areas (FIG. 10D) were identified in the examined section of IL2-CCR4 bispecific immunotoxin treated animal liver. In contrast, no tumor cell area (FIG. 10E) was identified in the examined section of the CCR4-IL2 bispecific immunotoxin treated animal liver. This histology data may explain why CCR4-IL2 immunotoxin lead to longer animal survival than IL2-CCR4 immunotoxin (FIG. 7).

1. A bispecific immunotoxin polypeptide chain comprising:
a toxin;
a human interleukin (IL-2); and
an anti-human CC Chemokine Receptor 4 (CCR4) antibody or fragment thereof, wherein the human IL-2 is linked to the anti-human CCR4 antibody.

2. The bispecific immunotoxin of claim 1, wherein the toxin is diphtheria toxin or variant.

3. The bispecific immunotoxin polypeptide chain of claim 2, wherein the anti-human CCR4 antibody is a single chain variable domain comprising the Complementarity Determining Regions encoded in SEQ ID NOs: 9 and 11.

4. The bispecific immunotoxin polypeptide chain of claim 2, wherein the toxin is linked to the human IL-2.

5. The bispecific immunotoxin polypeptide chain of claim 2, wherein the toxin is linked to the anti-human CCR4 antibody.

6. The bispecific immunotoxin polypeptide chain of claim 4, wherein the IL-2 is positioned at the c-terminus of the toxin.

7. A nucleic acid molecule encoding the bispecific immunotoxin of claim 6.

8. The nucleic acid molecule of claim 7, optimized for expression in a methylotropic yeast.

9. The nucleic acid molecule of claim 8, wherein the methylotropic yeast is Pichia pastoris.

10. The bispecific immunotoxin polypeptide chain of claim 5, wherein the CCR4 antibody is positioned at the c-terminus of the toxin.

11. A nucleic acid molecule encoding the bispecific immunotoxin of claim 10.

12. The nucleic acid molecule of claim 11, optimized for expression in a methylotropic yeast.

13. The nucleic acid molecule of claim 12, wherein the methylotropic yeast is Pichia pastoris.

14. A pharmaceutical composition comprising the bispecific immunotoxin of claim 4, and a physiologically acceptable carrier.

15. A pharmaceutical composition comprising the bispecific immunotoxin of claim 5, and a physiologically acceptable carrier.

16. A method of treating a subject in need thereof, the method comprising:
administering to the subject a therapeutically effective amount of the bispecific immunotoxin of claim 2.

17. The method of claim 16, wherein the subject suffers from cancer with CCR4+ and/or CD25+ tumors.

18. A method of depleting CD25-expressing or CCR4-expressing regulatory T cells in a subject, the method comprising administering to the subject an effective amount of the bispecific immunotoxin of claim 2.

19. A method of producing a single chain IL2-CCR4 bispecific immunotoxin, the method comprising:
expressing a codon-optimized nucleic acid molecule encoding the bispecific immunotoxin of claim 2 in a methylotropic yeast; and
substantially purifying the IL2-CCR4 bispecific immunotoxin, thereby producing the single chain IL2-CCR4 bispecific immunotoxin.

20. The method of claim 19, wherein the methylotropic yeast is Pichia pastoris.


SEQUENCE LISTING (20 sequences)

SEQ ID NO: 1 - DNA, 2391 bp, synthetic construct
  misc_feature 1..2391: note = CCR4-IL2 bispecific immunotoxin DNA sequence
  source 1..2391: mol_type = other DNA
gctggtgctgacgacgtcgtcgactcctccaagtccttcgtcatggagaacttcgcttcctaccacgggaccaagccaggttacgtcgactccatccagaagggtatccagaagccaaagtccggcacccaaggtaactacgacgacgactggaaggggttctactccaccgacaacaagtacgacgctgcgggatactctgtagataatgaaaacccgctctctggaaaagctggaggcgtggtcaaggtcacctacccaggtctgactaaggtcttggctttgaaggtcgacaacgctgagaccatcaagaaggagttgggtttgtccttgactgagccattgatggagcaagtcggtaccgaagagttcatcaagagattcggtgacggtgcttccagagtcgtcttgtccttgccattcgctgagggttcttctagcgttgaatatattaataactgggaacaggctaaggctttgtctgttgaattggagattaacttcgaaaccagaggtaagagaggtcaagatgcgatgtatgagtatatggctcaagcctgtgctggtaacagagtcagacgttctgttggttcctctttgtcctgtatcaacctagactgggacgtcatcagagacaagactaagaccaagatcgagtctttgaaagagcatggcccaatcaagaacaagatgtccgaatcccccgctaagaccgtctccgaggaaaaggccaagcaatacctagaagagttccaccaaaccgccttggagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatccatcgctttgtcctctttgatggttgctcaagctatcccattggtcggtgagttggttgacatcggtttcgctgcctacaacttcgtcgagtccatcatcaacttgttccaagtcgtccacaactcctacaaccgtccggcttactccccaggtcacaagacccaaccattcttgccatggggtggtggtggttctgacattgagttgactcaatctccatcttccttggctgtttctgctggtgagaaggttactatgtcttgtaagtcttcccaatctattttgtactcttccaaccaaaagaactacttggcttggtaccaacaaaagccaggtcaatctccaaagttgttgatttactgggcttctactagagagtctggtgttccagacagattcactggttctggttctggtactgacttcactttgactatttcttccgttcaagctgaggacttggctgtttactactgtcaccaatacttgtcttcctacactttcggtggtggtactaagttggagattaagggtggtggtggttctggtggtggtggatctggtggtggtggttctcaagttcaattgcaacaatctggtccagagttggttagaccaggtgcttctgttagaatttcttgtaaggcttctggttacactttcgcttcttactacattcaatggatgaagcaaagaccaggtcaaggtttggagtggattggttggattaacccaggtaacgttaacactaagtacaacgagaagttcaagggtaaggctactttgactgctgacaagtcttccactaccgcttacatgcaattgtcttccttgacttctgaggactctgctgtttacttctgtgctagatccacttactacagaccattggactactggggtcaaggtactaccgttactgtttcttccggtggtggtggttctggtggtggtggatccggtggtggtggttctgctccaacttcttcttctactaagaagactcaattgcaattggagcacttgttgttggacttgcaaatgattttgaacggtattaacaactacaagaacccaaagttgactagaatgttgactttcaagttctacatgccaaagaaggctactgagttgaagcacttgcaatgtttggaggaggaattgaagccattggaggaagttttgaacttggctcaatctaagaacttccacttgagaccaagagacttgatttctaacattaacgttattgttttggagttgaagggttctgagactactttcatgtgtgagtacgctgacgagactgctactattgttgagttcttgaacagatggattactttctgtcaatctattatctctactttgactcaccaccaccaccaccac

SEQ ID NO: 2 - protein, 797 aa, synthetic construct
  REGION 1..797: note = CCR4-IL2 bispecific immunotoxin amino acid sequence
AGADDVVDSSKSFVMENFASYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPAKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLPWGGGGSDIELTQSPSSLAVSAGEKVTMSCKSSQSILYSSNQKNYLAWYQQKPGQSPKLLIYWASTRESGVPDRFTGSGSGTDFTLTISSVQAEDLAVYYCHQYLSSYTFGGGTKLEIKGGGGSGGGGSGGGGSQVQLQQSGPELVRPGASVRISCKASGYTFASYYIQWMKQRPGQGLEWIGWINPGNVNTKYNEKFKGKATLTADKSSTTAYMQLSSLTSEDSAVYFCARSTYYRPLDYWGQGTTVTVSSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 3 - DNA, 2391 bp, synthetic construct
  misc_feature 1..2391: note = IL2-CCR4 bispecific immunotoxin DNA sequence
  source 1..2391: mol_type = other DNA
gctggtgctgacgacgtcgtcgactcctccaagtccttcgtcatggagaacttcgcttcctaccacgggaccaagccaggttacgtcgactccatccagaagggtatccagaagccaaagtccggcacccaaggtaactacgacgacgactggaaggggttctactccaccgacaacaagtacgacgctgcgggatactctgtagataatgaaaacccgctctctggaaaagctggaggcgtggtcaaggtcacctacccaggtctgactaaggtcttggctttgaaggtcgacaacgctgagaccatcaagaaggagttgggtttgtccttgactgagccattgatggagcaagtcggtaccgaagagttcatcaagagattcggtgacggtgcttccagagtcgtcttgtccttgccattcgctgagggttcttctagcgttgaatatattaataactgggaacaggctaaggctttgtctgttgaattggagattaacttcgaaaccagaggtaagagaggtcaagatgcgatgtatgagtatatggctcaagcctgtgctggtaacagagtcagacgttctgttggttcctctttgtcctgtatcaacctagactgggacgtcatcagagacaagactaagaccaagatcgagtctttgaaagagcatggcccaatcaagaacaagatgtccgaatcccccgctaagaccgtctccgaggaaaaggccaagcaatacctagaagagttccaccaaaccgccttggagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatccatcgctttgtcctctttgatggttgctcaagctatcccattggtcggtgagttggttgacatcggtttcgctgcctacaacttcgtcgagtccatcatcaacttgttccaagtcgtccacaactcctacaaccgtccggcttactccccaggtcacaagacccaaccattcttgccatggggtggtggtggttctgctccaacttcttcttctactaagaagactcaattgcaattggagcacttgttgttggacttgcaaatgattttgaacggtattaacaactacaagaacccaaagttgactagaatgttgactttcaagttctacatgccaaagaaggctactgagttgaagcacttgcaatgtttggaggaggaattgaagccattggaggaagttttgaacttggctcaatctaagaacttccacttgagaccaagagacttgatttctaacattaacgttattgttttggagttgaagggttctgagactactttcatgtgtgagtacgctgacgagactgctactattgttgagttcttgaacagatggattactttctgtcaatctattatctctactttgactggtggtggtggttctggtggtggtggatccggtggtggtggttctgacattgagttgactcaatctccatcttccttggctgtttctgctggtgagaaggttactatgtcttgtaagtcttcccaatctattttgtactcttccaaccaaaagaactacttggcttggtaccaacaaaagccaggtcaatctccaaagttgttgatttactgggcttctactagagagtctggtgttccagacagattcactggttctggttctggtactgacttcactttgactatttcttccgttcaagctgaggacttggctgtttactactgtcaccaatacttgtcttcctacactttcggtggtggtactaagttggagattaagggtggtggtggttctggtggtggtggatctggtggtggtggttctcaagttcaattgcaacaatctggtccagagttggttagaccaggtgcttctgttagaatttcttgtaaggcttctggttacactttcgcttcttactacattcaatggatgaagcaaagaccaggtcaaggtttggagtggattggttggattaacccaggtaacgttaacactaagtacaacgagaagttcaagggtaaggctactttgactgctgacaagtcttccactaccgcttacatgcaattgtcttccttgacttctgaggactctgctgtttacttctgtgctagatccacttactacagaccattggactactggggtcaaggtactaccgttactgtttcttcccaccaccaccaccaccac

SEQ ID NO: 4 - protein, 797 aa, synthetic construct
  REGION 1..797: note = IL2-CCR4 bispecific immunotoxin amino acid sequence
AGADDVVDSSKSFVMENFASYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPAKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLPWGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSDIELTQSPSSLAVSAGEKVTMSCKSSQSILYSSNQKNYLAWYQQKPGQSPKLLIYWASTRESGVPDRFTGSGSGTDFTLTISSVQAEDLAVYYCHQYLSSYTFGGGTKLEIKGGGGSGGGGSGGGGSQVQLQQSGPELVRPGASVRISCKASGYTFASYYIQWMKQRPGQGLEWIGWINPGNVNTKYNEKFKGKATLTADKSSTTAYMQLSSLTSEDSAVYFCARSTYYRPLDYWGQGTTVTVSSHHHHHH

SEQ ID NO: 5 - protein, 230 aa, synthetic construct
  REGION 1..230: note = Rearranged mouse variable heavy (VH) and variable light (VL) K domains of mAb1567
QVQLQQSGPELVRPGASVRISCKASGYTFASYYIQWMKQRPGQGLEWIGWINPGNVNTKYNEKFKGKATLTADKSSTTAYMQLSSLTSEDSAVYFCARSTYYRPLDYWGQGTTVTVSSDIELTQSPSSLAVSAGEKVTMSCKSSQSILYSSNQKNYLAWYQQKPGQSPKLLIYWASTRESGVPDRFTGSGSGTDFTLTISSVQAEDLAVYYCHQYLSSYTFGGGTKLEIK

SEQ ID NO: 6 - protein, 230 aa, synthetic construct
  REGION 1..230: note = Humanized variable heavy (VH) and variable light (VK) K domains of mAb1567
QVQLVQSGAEVKKPGASVKVSCKASGYTFASYYMHWMRQAPGQGLEWIGWINPGNVNTKYNEKFKGRATLTVDTSTNTAYMELSSLRSEDTAVYYCARSTYYRPLDYWGQGTLVTVSSDIVMTQSPDSLAVSLGERATINCKSSQSILYSSNQKNYLAWYQQKPGQSPKLLIYWASTRESGVPDRFSGSGSGTDFTLTISSLQAEDVAVYYCHQYLSSYTFGQGTKLEIK

SEQ ID NO: 7 - DNA, 1179 bp, synthetic construct
  misc_feature 1..1179: note = DT 390 DNA sequence
  source 1..1179: mol_type = other DNA
gctggtgctgacgacgtcgtcgactcctccaagtccttcgtcatggagaacttcgcttcctaccacgggaccaagccaggttacgtcgactccatccagaagggtatccagaagccaaagtccggcacccaaggtaactacgacgacgactggaaggggttctactccaccgacaacaagtacgacgctgcgggatactctgtagataatgaaaacccgctctctggaaaagctggaggcgtggtcaaggtcacctacccaggtctgactaaggtcttggctttgaaggtcgacaacgctgagaccatcaagaaggagttgggtttgtccttgactgagccattgatggagcaagtcggtaccgaagagttcatcaagagattcggtgacggtgcttccagagtcgtcttgtccttgccattcgctgagggttcttctagcgttgaatatattaataactgggaacaggctaaggctttgtctgttgaattggagattaacttcgaaaccagaggtaagagaggtcaagatgcgatgtatgagtatatggctcaagcctgtgctggtaacagagtcagacgttctgttggttcctctttgtcctgtatcaacctagactgggacgtcatcagagacaagactaagaccaagatcgagtctttgaaagagcatggcccaatcaagaacaagatgtccgaatcccccgctaagaccgtctccgaggaaaaggccaagcaatacctagaagagttccaccaaaccgccttggagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatccatcgctttgtcctctttgatggttgctcaagctatcccattggtcggtgagttggttgacatcggtttcgctgcctacaacttcgtcgagtccatcatcaacttgttccaagtcgtccacaactcctacaaccgtccggcttactccccaggtcacaagacccaaccattcttgccatgg

SEQ ID NO: 8 - DNA, 15 bp, synthetic construct
  misc_feature 1..15: note = Linker sequence
  source 1..15: mol_type = other DNA
ggtggtggtggttct

SEQ ID NO: 9 - DNA, 336 bp, synthetic construct
  misc_feature 1..336: note = anti-human CCR4 scFv VL
  source 1..336: mol_type = other DNA
gacattgagttgactcaatctccatcttccttggctgtttctgctggtgagaaggttactatgtcttgtaagtcttcccaatctattttgtactcttccaaccaaaagaactacttggcttggtaccaacaaaagccaggtcaatctccaaagttgttgatttactgggcttctactagagagtctggtgttccagacagattcactggttctggttctggtactgacttcactttgactatttcttccgttcaagctgaggacttggctgtttactactgtcaccaatacttgtcttcctacactttcggtggtggtactaagttggagattaag

SEQ ID NO: 10 - DNA, 45 bp, synthetic construct
  misc_feature 1..45: note = linker sequence
  source 1..45: mol_type = other DNA
ggtggtggtggttctggtggtggtggatctggtggtggtggttct

SEQ ID NO: 11 - DNA, 354 bp, synthetic construct
  misc_feature 1..354: note = anti-human CCR4 scFv VH
  source 1..354: mol_type = other DNA
caagttcaattgcaacaatctggtccagagttggttagaccaggtgcttctgttagaatttcttgtaaggcttctggttacactttcgcttcttactacattcaatggatgaagcaaagaccaggtcaaggtttggagtggattggttggattaacccaggtaacgttaacactaagtacaacgagaagttcaagggtaaggctactttgactgctgacaagtcttccactaccgcttacatgcaattgtcttccttgacttctgaggactctgctgtttacttctgtgctagatccacttactacagaccattggactactggggtcaaggtactaccgttactgtttcttcc

SEQ ID NO: 12 - DNA, 45 bp, synthetic construct
  misc_feature 1..45: note = linker sequence
  source 1..45: mol_type = other DNA
ggtggtggtggttctggtggtggtggatccggtggtggtggttct

SEQ ID NO: 13 - DNA, 417 bp, Homo sapiens
  source 1..417: mol_type = genomic DNA
gctccaacttcttcttctactaagaagactcaattgcaattggagcacttgttgttggacttgcaaatgattttgaacggtattaacaactacaagaacccaaagttgactagaatgttgactttcaagttctacatgccaaagaaggctactgagttgaagcacttgcaatgtttggaggaggaattgaagccattggaggaagttttgaacttggctcaatctaagaacttccacttgagaccaagagacttgatttctaacattaacgttattgttttggagttgaagggttctgagactactttcatgtgtgagtacgctgacgagactgctactattgttgagttcttgaacagatggattactttctgtcaatctattatctctactttgactcaccaccaccaccaccac

SEQ ID NO: 14 - DNA, 1179 bp, synthetic construct; identical to SEQ ID NO: 7
  misc_feature 1..1179: note = DT390
  source 1..1179: mol_type = other DNA

SEQ ID NO: 15 - DNA, 15 bp, synthetic construct
  misc_feature 1..15: note = linker sequence
  source 1..15: mol_type = other DNA
ggtggtggtggttct

SEQ ID NO: 16 - DNA, 399 bp, Homo sapiens
  source 1..399: mol_type = genomic DNA
gctccaacttcttcttctactaagaagactcaattgcaattggagcacttgttgttggacttgcaaatgattttgaacggtattaacaactacaagaacccaaagttgactagaatgttgactttcaagttctacatgccaaagaaggctactgagttgaagcacttgcaatgtttggaggaggaattgaagccattggaggaagttttgaacttggctcaatctaagaacttccacttgagaccaagagacttgatttctaacattaacgttattgttttggagttgaagggttctgagactactttcatgtgtgagtacgctgacgagactgctactattgttgagttcttgaacagatggattactttctgtcaatctattatctctactttgact

SEQ ID NO: 17 - DNA, 45 bp, synthetic construct; identical to SEQ ID NO: 12
  misc_feature 1..45: note = linker sequence
  source 1..45: mol_type = other DNA

SEQ ID NO: 18 - DNA, 336 bp, synthetic construct; identical to SEQ ID NO: 9
  misc_feature 1..336: note = anti-human CCR4 scFv VL
  source 1..336: mol_type = other DNA

SEQ ID NO: 19 - DNA, 45 bp, synthetic construct; identical to SEQ ID NO: 10
  misc_feature 1..45: note = linker sequence
  source 1..45: mol_type = other DNA

SEQ ID NO: 20 - DNA, 372 bp, synthetic construct
  misc_feature 1..372: note = anti-human CCR4 scFv VH
  source 1..372: mol_type = other DNA
caagttcaattgcaacaatctggtccagagttggttagaccaggtgcttctgttagaatttcttgtaaggcttctggttacactttcgcttcttactacattcaatggatgaagcaaagaccaggtcaaggtttggagtggattggttggattaacccaggtaacgttaacactaagtacaacgagaagttcaagggtaaggctactttgactgctgacaagtcttccactaccgcttacatgcaattgtcttccttgacttctgaggactctgctgtttacttctgtgctagatccacttactacagaccattggactactggggtcaaggtactaccgttactgtttcttcccaccaccaccaccaccac